(19) United States
(12) Patent Application Publication
(10) Pub. No.: US 2005/0261367 A1
(43) Pub. Date: Nov. 24, 2005

(54) Methods for treating dermatological and other health-related conditions in a patient

(76) Inventor: Howard Murad, Marina del Rey, CA (US)

Correspondence Address: KENYON & KENYON, 1500 K STREET NW, SUITE 700, WASHINGTON, DC, 20005, US

(21) Appl. No.: 11/090,567

(22) Filed: Mar. 28, 2005

Related U.S. Application Data
(60) Provisional application No. 60/556,870, filed on Mar. 29, 2004.
(60) Provisional application No. 60/599,555, filed on Aug. 9, 2004.

(51) Int. Cl. A61K 31/198; A61K 31/28

(52) U.S. Cl. 514/492; 514/561

(57) ABSTRACT

A method is provided for treating a dermatological condition in a patient. The method includes (a) measuring the intracellular water content of the patient to provide an initial intracellular water content measurement; (b) administering to the patient at least one active agent; (c) re-measuring the intracellular water content of the patient to provide a post-treatment intracellular water content measurement; and (d) repeating steps (b) and (c) until the post-treatment intracellular water content measurement is greater than the initial intracellular water content measurement. A method is also provided for treating other health-related conditions in a patient.

### CROSS-REFERENCE TO RELATED APPLICATIONS

This application claims the benefit of U.S. Provisional application 60/556,870, filed on Mar. 29, 2004 and U.S. Provisional application 60/599,555, filed on Aug. 9, 2004, both of which are incorporated herein by reference.

### STATEMENT REGARDING FEDERALLY SPONSORED RESEARCH OR DEVELOPMENT

Not applicable

### INCORPORATION-BY-REFERENCE OF MATERIAL SUBMITTED ON A COMPACT DISC

Not applicable

### SEQUENCE LISTING

Not applicable

### TECHNICAL FIELD

This application relates to methods for treating dermatological and other health-related conditions in a patient.

### BACKGROUND OF THE INVENTION

Human skin is a composite material of the epidermis and the dermis. The topmost part of the epidermis is the stratum corneum. This layer is the stiffest layer of the skin, as well as the one most affected by the surrounding environment. Below the stratum corneum is the internal portion of the epidermis. Below the epidermis is the dermis. The topmost layer of the dermis is the papillary dermis, which is made of relatively loose connective tissues that define the micro-relief of the skin. The reticular dermis, disposed beneath the papillary dermis, is tight, connective tissue that is spatially organized. The reticular dermis is also associated with coarse wrinkles. At the bottom of the dermis lies the subcutaneous layer.

The principal functions of the skin include protection, excretion, secretion, absorption, thermoregulation, pigmentogenesis, accumulation, sensory perception, and regulation of immunological processes. These functions are detrimentally affected by, for example, dryness, yeast, and structural changes in the skin, such as due to aging and excessive sun exposure.

Various pharmaceuticals have been used for the treatment or prevention of skin conditions, including skin moisturizing compositions that hydrate the skin. Some of these skin compositions include for example, those disclosed in U.S. Pat. No. 4,287,172 to Jacquit et al., U.S. Pat. No. 5,380,528 to Albani et al., U.S. Pat. No. 5,858,340 to Briggs et al., and U.S. Pat. No. 6,264,963 to Leifheit et al.

U.S. Pat. No. 5,804,168 discloses a pharmaceutical composition for the protection and prevention of skin damage to a patient resulting from exposure to sunlight. The composition comprises at least one antioxidant component in an amount sufficient to inhibit the formation of free radicals; at least one anti-inflammatory component in an amount sufficient to substantially inhibit the inflammation associated with exposure to sunlight; and at least one immunity boosting component to enhance the patient's immune response.

U.S. Pat. No. 5,804,594 to Howard Murad discloses a pharmaceutical composition for the prevention and treatment of skin conditions, including wrinkles, having a sugar compound that is converted to a glycosaminoglycan in the patient in an amount sufficient to thicken the skin, a primary antioxidant component in an amount sufficient to substantially inhibit the formation of collagenase and elastase, at least one amino acid component in an amount sufficient to assist in the thickening of the skin, and at least one transition metal component in an amount effective to bind collagen and elastic fibers and rebuild skin.

U.S. Pat. No. 5,962,517 discloses a pharmaceutical composition for the treatment of acne having an acne reduction component in an amount sufficient to reduce the redness and blemishes associated with acne.

U.S. Pat. Nos. 6,071,541 and 6,296,880 disclose pharmaceutical compositions and methods for the cleansing of skin to facilitate the prevention, treatment, and management of skin conditions, such as seborrheic dermatitis, psoriasis, folliculitis, rosacea, perioral dermatitis, acne, and impetigo. The composition includes a sufficient amount of an acidic component of a hydroxy acid or tannic acid, or a pharmaceutically acceptable salt thereof, to exfoliate a portion of the skin, a sufficient amount of stabilized hydrogen peroxide to facilitate cleansing of the skin without substantial irritation thereof, and an antimicrobial agent in an amount sufficient to inhibit or reduce microorganisms on the skin.

U.S. Pat. No. 6,194,452 discloses a non-irritating, stable pharmaceutical composition including a solution of at least one pharmaceutically acceptable silicone or oil and a source of pharmaceutically acceptable ascorbic acid. The solution is present in an amount sufficient to inhibit degradation of the ascorbic acid while facilitating the prevention or treatment of skin damage.

U.S. Pat. No. 6,630,163 to Howard Murad discloses methods for treating dermatological disorders, which include administering a therapeutically effective amount of at least one fruit extract in an amount sufficient to neutralize free radicals.

U.S. Pat. No. 6,673,374 to Howard Murad discloses pharmaceutical compositions and methods for treating inflammatory skin conditions. The compositions include hydrogen peroxide, one or more moisturizing agents, and an anti-inflammatory agent.

U.S. Pat. No. 6,676,977 to Howard Murad discloses compositions and methods for reducing or eliminating the appearance of cellulite. The method involves administering to a patient therapeutically effective amounts of a compound that is converted to a glycosaminoglycan in the patient in an amount sufficient to thicken the skin, a primary antioxidant component in an amount sufficient to substantially inhibit the formation of collagenase and elastase, at least one amino acid component in an amount sufficient to assist in the thickening of the skin, and at least one transition metal component in an amount effective to bind collagen and elastic fibers and thicken skin so as to reduce or eliminate the appearance of cellulite.

THE MURAD METHOD by Howard Murad, M. D. and Dianne Partie Lange (St. Martin's Press, April, 2003) discloses a holistic approach for treating the skin to slow the aging process.

Despite these references, there remains a need for improved methods of treating and maintaining healthier skin and treating other health related disorders. Citation of any reference in this section of the application is not to be construed as an admission that such reference is prior art to the present application.

### SUMMARY OF THE INVENTION

The present invention encompasses a method for treating a dermatological condition in a patient comprising (1) measuring the intracellular water content of the patient to provide an initial intracellular water content measurement; (2) administering to the patient at least one active agent; (3) re-measuring the intracellular water content of the patient to provide a post-treatment intracellular water content measurement; and (4) repeating the second and third steps until the post-treatment intracellular water content measurement is greater than the initial intracellular water content measurement. The at least one active agent can be administered topically or orally.

In one embodiment, the post-treatment intracellular water content measurement is at least about 0.25 percentage units greater than the initial intracellular water content measurement, e.g., the initial intracellular water content measurement is 25% and the post-treatment intracellular water content is 25.25%. In one embodiment, the post-treatment intracellular water content is at least about 1 percentage unit greater than the initial intracellular water content measurement. In one embodiment, the post-treatment intracellular water content measurement is at least about 3 percentage units greater than the initial intracellular water content measurement. In one embodiment, the post-treatment intracellular water content measurement is at least about 5 percentage units greater than the initial intracellular water content measurement. In one embodiment, the post-treatment intracellular water content measurement is at least about 10 percentage units greater than the initial intracellular water content measurement. In one embodiment, the post-treatment intracellular water content measurement is at least about 15 percentage units greater than the initial intracellular water content measurement. In extreme cases, the post-treatment intracellular water content measurement can be at least about 25 percentage units greater than the initial intracellular water content measurement.

In one embodiment, the active agent is administered to the patient at least daily for at least about 1 day before re-measuring the intracellular water content of the patient's skin. In one embodiment, the active agent is administered to the patient at least daily for at least about 7 days before re-measuring the intracellular water content of the patient's skin. In one embodiment, the active agent is administered to the patient at least daily for at least about 14 days before re-measuring the intracellular water content of the patient's skin. In one embodiment, the active agent is administered to the patient at least daily for at least about 28 days before re-measuring the intracellular water content of the patient's skin. In one embodiment, the active agent is administered to the patient at least daily for at least about 35 days before re-measuring the intracellular water content of the patient's skin.

The methods of the invention can further comprise taking a photographic image of the patient's skin before obtaining the initial intracellular water content measurement and after obtaining the post-treatment intracellular water content measurement to record a first and second photograph of the dermatological condition and comparing the first photograph and the second photograph to evaluate whether a further increase in the intracellular water content of the patient's skin is needed to improve the dermatological condition.

The at least one active agent of the invention can comprise one or more of lecithin, phosphatidylcholine, or choline. The at least one active agent can also comprise a compound that is converted to a glycosaminoglycan in the patient, a hydrophillic moisturizing agent, a hydrophobic moisturizing agent, an anti-inflammatory agent, a primary antioxidant component, an amino acid component, or a transition metal compound.

The method can further comprise administering to the patient at least two active agents.

The invention further comprises a method for treating a health-related condition in a patient comprising: (1) measuring the intracellular water content of the patient to provide an initial intracellular water content measurement; (2) administering to the patient at least one active agent; (3) re-measuring the intracellular water content of the patient to provide a post-treatment intracellular water content measurement; and (4) repeating the third and fourth steps until the post-treatment intracellular water content measurement is greater than the initial intracellular water content measurement.

The health-related conditions which are treated by the methods of the invention can include, but are not limited to, high blood pressure, hair loss, liver disorders, heart disorders, lung disorders, kidney disorders, disorders of the nervous system, brain disorders, disorders of the digestive system, disorders of the reproductive system, and cancer.

### BRIEF DESCRIPTION OF THE DRAWINGS

For a more complete understanding of the present invention and for further advantages thereof, reference is made to the following drawing:

FIG. 1 generally depicts the method of the invention in the form of a flow diagram.

### DETAILED DESCRIPTION OF THE INVENTION

The present invention is directed to a method for treating a dermatological condition in a patient comprising: (a) measuring the intracellular water content of the patient to provide an initial intracellular water content measurement; (b) administering to the patient at least one active agent; (c) re-measuring the intracellular water content of the patient to provide a post-treatment intracellular water content measurement; and (d) repeating steps (b) and (c) until the post-treatment intracellular water content measurement is greater than the initial intracellular water content measurement.

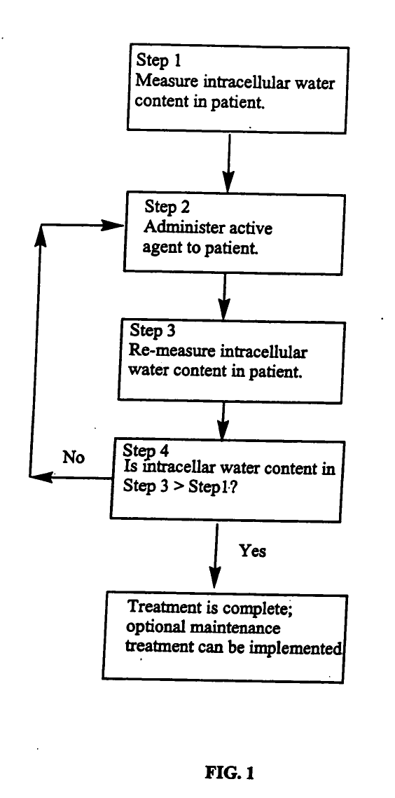

FIG. 1 represents the method of the invention in the form of a flow diagram. In the first step, the intracellular water content of a patient is measured. In the second step, at least one active agent is administered. In the third step, the intracellular water content of the patient is re-measured. In the fourth step, an assessment is made whether the intracellular water content from step 3 is greater than that in step 1. If the intracellular water content in step 3 is not greater than that in step 1, steps 2 and 3 are repeated until the intracellular water content in step 3 is greater than the intracellular water content in step 1. If the intracellular water content in step 3 is greater than the intracellular water content in step 1, the method is complete with the exception of optional maintenance therapy. In a preferred embodiment, optional maintenance treatment comprises continuing to administer to the patient one or more active agents to maintain the intracellular water content at a level higher than the initial intracellular water content measurement, or to further increase intracellular water content of the patient.

In one embodiment, the method further comprises (a1) measuring the extracellular water content of the patient before administering the at least one active agent to the patient to provide an initial extracellular water content measurement and (c1) then re-measuring the extracellular water content after administering the at least one active agent to the patient to provide a post-treatment extracellular water content measurement; and (d1) repeating steps (a1) and (c1) until the post-treatment extracellular water content measurement is less than the initial extracellular water content measurement.

Without wishing to be bound by theory, it is believed that an increase in intracellular water content and a decrease in extracellular water content provides a healthier and more youthful appearing skin.

In another embodiment, the method further comprises (a2) measuring the basal metabolic rate of the patient before administering the at least one active agent to the patient to provide an initial basal metabolic rate measurement and (c2) then re-measuring the basal metabolic rate after administering the at least one active agent to the patient to provide a post-treatment basal metabolic rate measurement; and (d2) repeating steps (a2) and (c2) until the post-treatment basal metabolic rate measurement is greater than the initial basal metabolic rate measurement.

Without wishing to be bound by theory, it is believed that an increase in intracellular water content and an increase in basal metabolic rate provides a healthier and more youthful appearing skin.

In yet another embodiment, the method further comprises (a1) measuring the extracellular water content of the patient before administering the at least one active agent to the patient to provide an initial extracellular water content measurement and (a2) measuring the basal metabolic rate of the patient before administering the at least one active agent to the patient to provide an initial basal metabolic rate measurement; then (c1) re-measuring the extracellular water content after administering the at least one active agent to the patient to provide a post-treatment extracellular water content measurement and (c2) re-measuring the basal metabolic rate after administering the at least one active agent to the patient to provide a post-treatment basal metabolic rate measurement; and (d1) repeating steps (a1) and (c1) until the post-treatment extracellular water content measurement is less than the extracellular water content measurement and (d2) repeating steps (a2) and (c2) until the post-treatment basal metabolic rate measurement is greater than the initial basal metabolic rate measurement.

The intracellular water content, extracellular water content, and total water content can be measured by any method well known to those skilled in the art. For example, the intracellular water content, extracellular water content, basal metabolic rate, and total water content can be measured using a bioelectrical impedance analyzer, such as the Bioelectrical Body Composition Analyzer commercially available from RJL systems of Clinton Township, Michigan.

The term “intracellular water content” as used herein means the water in the human body occurring within the cells of the body.

The term “extracellular water content” as used herein means the water in the human body found outside of the cells of the body and between the cells in a tissue.

The term “total water content” as used herein means all of the water present in the human body, i.e., the sum of the intracellular water content and extracellular water content.

The term “basal metabolic rate,” as used herein means the number of calories your body burns at rest to maintain normal body functions.

The term “cell membrane” as used herein means the structure enveloping a cell, enclosing the cytoplasm, and forming a selective permeability barrier between the cytoplasm and the outside of the cell.

The term “therapeutically effective amount,” as used herein, means that amount of the active agent that provides a therapeutic benefit in the treatment, prevention, or management of one or more health-related conditions, e.g., dermatological conditions.

The term “health-related condition” as used herein means an undesirable physical state in a patient, including, but not limited to, dermatological conditions, disorders (e.g., lung disorders, heart disorders, kidney disorders, liver disorders, brain disorders, disorders of the nervous system, and disorders of the reproductive system), diseases (e.g., cancer), hair loss, and high blood pressure.

The term “disorder” as used herein means a disturbance of function, structure, or both, resulting from a genetic or embryonic failure in development or from exogenous factors such as poison, trauma, or disease.

The term “disease” as used herein means an interruption, cessation, or disorder of a body function, system, or organ.

Without wishing to be bound by theory, Applicant believes that increasing a patient's intracellular water content can provide healthier and more youthful appearing skin. More critical than total water content to the maintenance of healthy skin is the intracellular water content. Applicant has discovered that high intracellular water content correlates with healthier skin. Applicant's invention is directed to a method of treating dermatological conditions. This method comprises: (a) measuring the intracellular water content of the patient to provide an initial intracellular water content measurement; (b) administering to the patient at least one active agent; (c) re-measuring the intracellular water content of the patient to provide a post-treatment intracellular water content measurement; and (d) repeating steps (b) and (c) until the post-treatment intracellular water content measurement is greater than the initial intracellular water content measurement.

The active agents administered in this method improve the ability of the patient's cells to retain intracellular water. In one embodiment, this is accomplished by strengthening a patient's cell membranes to reduce loss of intracellular water by the skin cells. Without wishing to be bound by theory, Applicant believes that the aging process and environmental exposure cause damage to the cell membrane of skin cells that results in the loss of intracellular water by the skin cells. Accordingly, by inhibiting or minimizing damage to the cell membrane and/or repairing and strengthening the cell membrane of skin cells, the loss of intracellular water is reduced and the skin is healthier and has a more youthful appearance.

The total water content of a person's body decreases as a person ages. For example, at birth, about 75% of a human's weight is water. As humans age, however, their total water content, and in particular the intracellular water content, decreases, approaching a total water content in the range of about 36-64% for adults. Although intracellular water content can vary by age, sex and other factors, a person's intracellular water content is generally equal to or slightly greater than their extracellular water content. For an adult, both intracellular water content and extracellular water content are commonly in the range of about 18-32% by weight. However, these values for adults are more typically in the range of 21-26% by weight.

Without wishing to be bound by theory, Applicant believes that the loss of intracellular water content leaves cells and other areas of the body weakened and less effective. When cells are not sufficiently hydrated they cannot function at their optimal level. This leads to much of the tissue damage associated with aging. By administering the appropriate active agent to a patient, the breakdown of skin cell membranes can be prevented, repaired, and even reversed. Applicant has discovered that this prevention, repair, and reversal is correlated with intracellular water content. Applicant has, moreover, discovered that an increase in intracellular water content is an indication of healthier and better functioning cells.

Aging of the skin has many causes, all of which result in intracellular water loss. A representative example is aging of the skin caused by free radical damage. Without wishing to be bound by theory, Applicant believes that factors including, but not limited to, smoking, poor diet, stress, and exposure to pollution and the sun all create free radicals, which in turn can damage the skin. One of the most significant ways in which free radicals harm the skin is by reacting with the lipids in the cell membrane. Ultimately this breaks down the cell membrane, allowing water to escape from the cell and leading to cell injury and premature cell death. A sign of such damage would be a low value for a person's intracellular water content. Accordingly, an increase in intracellular water content is correlated with reduced damage and healthier skin.

Dermatological conditions that can be treated by the methods of this invention include, but are not limited to, conditions anywhere on the skin caused by aging or extrinsic factors such as sunlight, radiation, air pollution, wind, cold, dampness, heat, chemicals, smoke, and smoking. Additional dermatological conditions which can be treated by the methods of this invention include, but are not limited to, dry skin; dandruff; warts; keratosis; pruritus; age spots; reduced skin moisture; spider veins; senile purpura; lentigines; melasmas; deepening of skin lines; blotches; wrinkles; blemished skin; nodules; atrophy; precancerous lesions; elastotic changes characterized by leathery, course, rough, dry and yellowish skin; telangiecatic skin; hyperpigmented skin; hyperkeratotic skin; nail infections; inflammatory dermatoses; seborrheic dermatitis; nummular dermatitis; contact dermatitis; atopic dermatitis; exfoliative dermatitis; perioral dermatitis; stasis dermatitis; psoriasis; impetigo; erysipelas; paronychia; erythrasma; eczema; psoriasis; folliculitis; rosacea; acne; cellulite; microbial infections; and damage to hair including, but not limited to, hair breakage, weathering damage, and thinning of hair.

Applicant has also discovered that increasing intracellular water content correlates with benefits to other tissues in the body, e.g., organs (e.g., heart, lungs, kidneys and brain) and connective tissue (e.g., blood vessels, nerves, ligaments, tendons). Without wishing to be bound by theory, Applicant believes that all health-related conditions are associated with a decrease in intracellular water content. Such health-related conditions include, but are not limited to dermatological conditions, disorders of the major organs (such as liver disorders, kidney disorders, heart disorders, lung disorders, and brain disorders), diseases, high blood pressure, the adverse effects of menstruation, and hair loss.

Without wishing to be bound by theory, Applicant believes an increase in intracellular water content is a defining characteristic associated with improved health. Dehydrated cells perform their functions less efficiently than hydrated cells. The cells that make up our liver, brain, heart, lungs, and every other organ of our body do not perform as efficiently if they are not sufficiently hydrated. Cells with less than a sufficient amount of water are likely to take far longer to recover from illness and infection then cells that are sufficiently hydrated. Accordingly, an individual whose cells are sufficiently hydrated is capable of recovering from illness or infection more quickly than if their cells were not sufficiently hydrated. Thus, an increase in intracellular water content not only correlates with strengthening the membrane of skin cells so that they are better able to maintain water and to provide a healthier skin with a more youthful appearances but also correlates with strengthening the membrane of every other cell of the body so that these other cells are also better able to maintain water. Moreover, Applicant believes that strengthening a patient's cell membranes improves a cells ability to absorb extracellular water, i.e., water in the human body found outside of the cells of the body and between the cells in a tissue. High levels of extracellular water, known as edema, is unhealthy. Accordingly, overall improved health is also correlated with a decrease in extracellular water content.

Accordingly, the invention further relates to a method of treating a health-related condition in a patient comprising (a) measuring the patient's intracellular water content to provide an initial intracellular water content measurement, (b) administering to the patient at least one active agent, (c) re-measuring the patient's intracellular water content to provide a post-treatment intracellular water content measurement, and (d) repeating steps (b) and (c) until the post-treatment intracellular water content measurement is greater than the initial intracellular water content measurement. Health-related conditions that can be treated by the method of the invention include, but are not limited to, high blood pressure, the adverse effects of menstruation, abnormal sensation, diabetes, and hair loss.

Increases in intracellular water content experienced in the method to treat a health-related condition should be similar in magnitude to the increases in intracellular water content experienced in the method to treat a dermatological condition.

In one embodiment, the method of treating a health-related condition in a patient further comprises (a1) measuring the extracellular water content of the patient before administering the at least one active agent to the patient to provide an initial extracellular water content measurement and (c1) then re-measuring the extracellular water content after administering the at least one active agent to the patient to provide a post-treatment extracellular water content measurement; and (d1) repeating steps (a1) and (c1) until the post-treatment extracellular water content measurement is less than the initial extracellular water content measurement.

Decreases in extracellular water content experienced in the method to treat a health-related condition should be similar in magnitude to the decreases in extracellular water content experienced in the method to treat a dermatological condition.

In another embodiment, the method of treating a health-related condition in a patient further comprises (a2) measuring the basal metabolic rate of the patient before administering the at least one active agent to the patient to provide an initial basal metabolic rate measurement and (c2) then re-measuring the basal metabolic rate after administering the at least one active agent to the patient to provide a post-treatment basal metabolic rate measurement; and (d2) repeating steps (a2) and (c2) until the post-treatment basal metabolic rate measurement is greater than the initial basal metabolic rate measurement.

Increases in basal metabolic rate experienced in the method to treat a health-related condition should be similar in magnitude to the increases in basal metabolic rate experienced in the method to treat a dermatological condition.

In yet another embodiment, the method of treating a health-related condition in a patient further comprises (a1) measuring the extracellular water content of the patient before administering the at least one active agent to the patient to provide an initial extracellular water content measurement and (a2) measuring the basal metabolic rate of the patient before administering the at least one active agent to the patient to provide an initial basal metabolic rate measurement; then (c1) re-measuring the extracellular water content after administering the at least one active agent to the patient to provide a post-treatment extracellular water content measurement and (c2) re-measuring the basal metabolic rate after administering the at least one active agent to the patient to provide a post-treatment basal metabolic rate measurement; and (d1) repeating steps (a1) and (c1) until the post-treatment extracellular water content measurement is less than the extracellular water content measurement and (d2) repeating steps (a2) and (c2) until the post-treatment basal metabolic rate measurement is greater than the initial basal metabolic rate measurement.

For example, in one embodiment, the health-related condition is high blood pressure. Without wishing to be bound by theory, Applicant believes that an increase in intracellular water content is indicative of strengthened blood vessels that result in lower blood pressure in a patient.

As another representative example, the health-related condition is the sense of touch, especially in a patient who has abnormal sensation. Without wishing to be bound by theory, Applicant believes that an increase in intracellular water content is indicative of improved health of nerve tissue that can result in an improved sense of touch.

As another representative example, the health-related condition is hair loss. Without wishing to be bound by theory, Applicant believes that an increase in intracellular water content is indicative of improved hair growth and a decrease in hair loss.

As another representative example, the health-related condition is the adverse effects of menstruation. Without wishing to be bound by theory, Applicant believes that an increase in intracellular water content is indicative of less painful menstruation.

As another representative example, the health-related condition is the erectile dysfunction. Without wishing to be bound by theory, Applicant believes that an increase in intracellular water content is indicative of a decrease in erectile dysfunction.

In one embodiment, the methods of the invention are provided for treating free radical damage to the skin.

In another embodiment, the methods of the invention are provided for treating warts.

In another embodiment, the methods of the invention are provided for treating cellulitis.

In another embodiment, the methods of the invention are provided for treating keratosis.

In another embodiment, the methods of the invention are provided for treating pruritus.

In another embodiment, the methods of the invention are provided for treating age spots.

In another embodiment, the methods of the invention are provided for treating reduced skin moisture.

In another embodiment, the methods of the invention are provided for treating spider veins.

In another embodiment, the methods of the invention are provided for treating senile purpura.

In another embodiment, the methods of the invention are provided for treating lentigines.

In another embodiment, the methods of the invention are provided for treating melasmas.

In another embodiment, the methods of the invention are provided for treating deepening of skin lines.

In another embodiment, the methods of the invention are provided for treating blotches.

In another embodiment, the methods of the invention are provided for treating wrinkles.

In another embodiment, the methods of the invention are provided for treating blemished skin.

In another embodiment, the methods of the invention are provided for treating nodules.

In another embodiment, the methods of the invention are provided for treating atrophy.

In another embodiment, the methods of the invention are provided for treating precancerous lesions.

In another embodiment, the methods of the invention are provided for treating elastotic changes characterized by leathery, course, rough, dry and yellowish skin.

In another embodiment, the methods of the invention are provided for treating telangiecatic skin.

In another embodiment, the methods of the invention are provided for treating hyperpigmented skin.

In another embodiment, the methods of the invention are provided for treating nail infections.

In another embodiment, the methods of the invention are provided for treating inflammatory dermatoses.

In another embodiment, the methods of the invention are provided for treating seborrheic dermatitis.

In another embodiment, the methods of the invention are provided for treating nummular dermatitis.

In another embodiment, the methods of the invention are provided for treating contact dermatitis.

In another embodiment, the methods of the invention are provided for treating atopic dermatitis.

In another embodiment, the methods of the invention are provided for treating exfoliative dermatitis.

In another embodiment, the methods of the invention are provided for treating perioral dermatitis.

In another embodiment, the methods of the invention are provided for treating stasis dermatitis.

In another embodiment, the methods of the invention are provided for treating erysipelas.

In another embodiment, the methods of the invention are provided for treating paronychia.

In another embodiment, the methods of the invention are provided for treating eczema.

In another embodiment, the methods of the invention are provided for treating erythrasma.

In another embodiment, the methods of the invention are provided for treating-psoriasis.

In another embodiment, the methods of the invention are provided for treating impetigo.

In another embodiment, the methods of the invention are provided for treating folliculitis.

In another embodiment, the methods of the invention are provided for treating rosacea.

In another embodiment, the methods of the invention are provided for treating acne.

In another embodiment, the methods of the invention are provided for treating microbial infections of the skin.

In another embodiment, the methods of the invention are provided for treating damage to hair including, but not limited to, hair breakage, weathering damage, and thinning of hair.

In another embodiment, the methods of the invention are provided for treating high blood pressure.

In another embodiment, the methods of the invention are provided for treating the adverse effects of menopause.

In another embodiment, the methods of the invention are provided for treating a liver disorder. Examples of liver disorders include, but are not limited to, hepatitis, cirrhosis, and alcohol-related fatty liver disease.

In another embodiment, the methods of the invention are provided for treating a heart disorder. Examples of heart disorders include, but are not limited to, angina, coronary artery disease, congestive heart failure, and endocarditis.

In another embodiment, the methods of the invention are provided for treating a lung disorder. Examples of lung disorders include, but are not limited to, pneumonia, bronchitis, emphysema, and asthma.

In another embodiment, the methods of the invention are provided for treating a kidney disorder. Examples of kidney disorders include, but are not limited to diabetic nephropathy and renal artery stenosis.

In another embodiment, the methods of the invention are provided for treating a disorder of the central nervous system and brain. Examples of disorders of the central nervous system and brain include, but are not limited to, attention deficit disorder, migraines and spinal cord diseases.

In another embodiment, the methods of the invention are provided for treating a disorder of the digestive system. Examples of disorders of the digestive system include, but are not limited to, ulcers, gastritis, and irritable bowel syndrome.

In another embodiment, the methods of the invention are provided for treating a disorder of the reproductive system. Examples of disorders of the reproductive system include, but are not limited to, erectile dysfunction, prostrate problems in males and endometriosis in females.

In another embodiment, the methods of the invention are provided for treating edema. For example, the methods of the invention are useful for treating lymphodema, i.e., edema of the arms, that is commonly associated with removal of lymph nodes, which is routinely performed as part of surgical treatment for breast cancer.

In another embodiment, the methods of the invention are provided for treating cancer.

Applicant realizes that an increase in intracellular water content may not cure all health-related conditions. However, increasing the intracellular water content in a patient according to the methods of the invention will facilitate the effectiveness of other treatment regimens. In other words, increasing the intracellular water content makes other treatment regimens more effective at treating a health related disorder.

In general, however, increasing the intracellular water content in a patient according to the methods of the invention results in a general improvement of the patients health.

Applicant's method for treating dermatological or other health-related conditions comprises measuring the intracellular water content to provide an initial intracellular water content of a patient, administering to the patient one or more active agents to increase the intracellular water content of the patient, re-measuring the intracellular water content of the patient to provide a post-treatment intracellular water content measurement, and continuing to administer the one or more active agents and re-measuring the intracellular water content until the post-treatment intracellular water content is greater than the initial intracellular water content.

In one embodiment, the method encompasses setting up clinics, staffed with a cosmetologist or dermatologist, who treats patients who visit the clinic using the above-described methods to increase the intracellular water content to treat a dermatological condition or health related condition in the patient.

In another embodiment, the invention is directed to a method of assessing a composition for its utility at treating a dermatological condition or other health related condition, i.e., its utility as an active agent. In this embodiment, the method comprises (a) measuring the intracellular water content of a patient to provide an initial intracellular water content measurement; (b) administering to the patient an agent to increase the intracellular water content of the patient; (c) re-measuring the intracellular water content of the patient to provide a post-treatment intracellular water content measurement; and (d) assessing whether the agent effectively increases the intracellular water content of the patient based upon the difference between the initial and the post-treatment intracellular water content. If the agent causes an increase in the intracellular water content, that is an indication that the agent can be used to treat a dermatological condition or health-related condition, i.e., it is an active agent.

In another embodiment, the invention is directed to a method of determining the optimum dose of an active agent to effectively increase a patient's intracellular water content. In this embodiment, the method comprises (a) measuring the intracellular water content of a patient to provide an initial intracellular water content measurement; (b) administering to the patient a dose of the active agent to increase the intracellular water content of the patient; (c) re-measuring the intracellular water content of the patient to provide a post-treatment intracellular water content measurement; (d) assessing whether the dose of the active agent effectively increased the intracellular water content of the patient based upon the difference between the initial and the post-treatment intracellular water content; and (e) repeating steps (b) through (d) using different doses until an optimum dose of the active agent for increasing the intracellular water content of the patient is determined.

Active agents useful in the methods of the present invention are generally administered in a pharmaceutical composition comprising the active agent and a pharmaceutically acceptable carrier or excipient.

The active agents used in the methods of the invention include, but are not limited to, those agents that strengthen cell membranes, agents that protect cell membranes from damage, and agents that repair damaged cell membranes. Active agents useful in the methods of the invention also include agents that prevent, minimize, or inhibit damage to cell membranes from the aging process. Active agents useful in the methods of the invention also include agents that provide moisture to the cells or increase the cells ability to absorb moisture, i.e., moisturizing agents.

Representative active agents that strengthen or repair damaged cell membranes include, but are not limited to, lecithin; phosphatidylcholine; choline; essential fatty acids including, but not limited to, gamma linoleic acid (GLA) and other fish oils that may include, for example the omega-3, omega-6, omega-9 oils and/or linoleic acid; and other lipids that are essential parts of the cell membrane, including panthenol.

In one embodiment, the active agent is lecithin.

In one embodiment, the active agent is phosphatidylcholine.

In one embodiment, the active agent is choline.

In one embodiment, the active agent is a combination of two agents selected from lecithin, phosphatidylcholine, or choline.

In one embodiment, the active agent is all three of lecithin, phosphatidylcholine, and choline.

Lecithin, phosphatidycholine, or choline can be included in a pharmaceutical composition for use in the method of the invention in an amount ranging from about 0.5 to 50 weight percent, preferably in an amount ranging from about 1 to 40 weight percent, and more preferably in an amount ranging from about 2 to 30 weight percent of a pharmaceutical composition. These agents are essential building blocks of the lipid layer surrounding the cytoplasm of the cells and forming the foundation of the cell membrane. Without wishing to be bound by theory, it is believed that administering these agents to the patient will fortify cell membranes thereby increasing the cells ability to retain intracellular water.

In one embodiment, the active agent useful in the pharmaceutical composition used in the method of the invention is Youth Builder, commercially available from Murad Inc., El Segundo, Calif.

In one embodiment, the active agent useful in the pharmaceutical composition used in the method of the invention is Wet Suit, commercially available from Murad Inc., El Segundo, Calif.

In one embodiment, the active agent useful in a pharmaceutical composition used in the method of the invention is a moisturizing agent. “Moisturizing agent,” as used herein, is used to include any agent that facilitates hydration of the skin by inhibiting or preventing loss of water from the skin, absorbing water from the atmosphere to hydrate the skin, or enhanceing the skin's own ability to absorb water directly from the atmosphere, or a combination thereof. Moisturizing agents also minimize or prevent the skin from drying and cracking; cracked skin is more susceptible to environmental factors that generate free radicals, which are believed to cause damage to the cell membrane of the skin cells. Without wishing to be bound by theory it is also believed that the moisturizing agent also improves the skin's ability to absorb other active agents used in the methods of the invention. Suitable moisturizing agents include, but are not limited to, hydrophobic agents, and hydrophilic agents, or combinations thereof. Moisturizers, when used, are typically present in an amount ranging from about 0.01 to 20 weight percent, preferably about 0.05 to 10 weight percent, more preferably from about 0.1 to 5 weight percent of the pharmaceutical composition.

Moisturizing agents that are hydrophobic agents include, but are not limited to, ceramide, borage oil (linoleic acid), tocopherol (Vitamin E), tocopherol linoleate, dimethicone, glycerine, and mixtures thereof. Hydrophobic agents, when present, are believed to moisturize the skin by inhibiting or preventing the loss of water from the skin. The hydrophobic agent, when present, is typically present in an amount ranging from about 0.01 to 20 weight percent, preferably from about 0.05 to 15 weight percent, and more preferably from about 0.1 to 5 weight percent of the pharmaceutical composition.

Moisturizing agents that are hydrophilic agents include, but are not limited to, hyaluronic acid, sodium peroxylinecarbolic acid (sodium PCA), wheat protein (e.g., laurdimonium hydroxypropyl hydrolyzed wheat protein), hair keratin amino acids, and mixtures thereof. Sodium chloride may also be present, particularly when hair keratin amino acids are included as a moisturizer. Hydrophilic agents, when present, are believed to moisturize the skin by absorbing moisture from the atmosphere to hydrate or facilitate hydration of the skin. The hydrophilic agent, when present, is typically present in an amount ranging from about 0.01 to 20 weight percent, preferably from about 0.05 to 15 weight percent, and more preferably from about 0.1 to 5 weight percent of the pharmaceutical composition.

Other moisturizing agents that hydrate the skin and are useful in the compositions and methods of the present invention include, but are not limited to, panthenol; primrose oil; gamma linoleic acid (GLA) and other fish oils that may include, for example, the omega-3, omega-6 and omega-9 oils and/or linoleic acid; and flax seed oil. Preferably, these moisturizing agents are administered orally.

Active agents useful in the methods of the invention, some of which may increase intracellular water content through mechanisms other than strengthening the cell membrane, include, but are not limited to, anti-inflammatory agents, amino acids, antioxidants, vitamins, minerals, transition metals, and compounds that are converted to a glycosaminoglycan in a patient.

The active agent can be a compound that is converted to a glycosaminoglycan. Any compound that is converted to a glycosaminoglycan in the patient can be used as the active agent in the methods of the invention. Typically, the compound that is converted to a glycosaminoglycan in the patient is a sugar or a derivative of a sugar. Derivatives of sugars include, but are not limited to carboxylate esters of sugars, phosphate esters of sugars, sulfate esters of sugars, and aminoglycans. Typically, the compound that is converted to glycosaminoglycans in the patient is N-acetylglucosamine, or a pharmaceutically acceptable salt or ester thereof. Preferably the compound that is converted to glycosaminoglycans in the patient is N-acetylglucosamine. Without wishing to be bound by theory, it is thought that glycosaminoglycans help to pull water into the dermis. The N-acetylglucosamine is present in an amount ranging from about 5 to 30 weight percent, preferably 8 to 27 weight percent, and more preferably 12 to 24 weight percent of the pharmaceutical composition. A unit oral dose of N-acetylglucosamine is typically about 40 mg to 250 mg, preferably about 60 to 200, and more preferably about 100 mg to 200 mg.

The active agent can be an anti-inflammatory agent. Without wishing to be bound by theory, it is believed that inflammation has a destructive effect on cell membranes and creates an excessive amount of free radicals, which contributes to cell membrane damage and therefore increased loss of intracellular water. In one embodiment the anti-inflammatory agent is a steroidal anti-inflammatory. Suitable steroidal anti-inflammatory agents useful in the methods of the invention include, but are not limited to, the corticosteroids such as, but not limited to, hydrocortisone, fluocinolone acetonide, halcinonide, halobetasol propionate, clobetasol propionate, betamethasone dipropionate, betamethasone valerate, and triamcinolone acetonide.

In another embodiment, the anti-inflammatory agent is a non-steroidal anti-inflammatory agent. Examples of suitable non-steroidal anti-inflammatory agents useful in the methods of the invention include, but are not limited to, aspirin, ibuprofen, ketoprofen, and naproxen. These anti-inflammatory agents are preferably administered orally. Other non-steroidal anti-inflammatory agents useful in the methods of the invention include, but are not limited to aloe vera gel, aloe vera, licorice extract, pilewort, Canadian willow root, and zinc, and allantoin. Allantoin is a preferred non-steroidal anti-inflammatory agent. The anti-inflammatory agents are used in an amount sufficient to inhibit or reduce inflammation, preferably in an amount ranging from about 0.02 to 2 weight percent, preferably from about 0.1 to 1.5 weight percent, and more preferably from about 0.2 to 1 weight percent of the pharmaceutical composition. Arnica Montana (a healing herb) and vitamin K can also be used as the anti-inflammatory. Arnica Montana facilitates skin healing and acts as an antiseptic and local anti-inflammatory, and, when used, is typically present in an amount from about 0.1 to 2 weight percent, preferably about 0.2 to 1 weight percent of the pharmaceutical composition. The Vitamin K inhibits or suppresses inflammation and bruising (i.e., acts as an anti-inflammatory and anti-bruising agent) and, when used, is typically present in an amount from about 0.01 to 1 weight percent, preferably from about 0.1 to 0.5 weight percent of the pharmaceutical composition. When used to treat a dermatological condition, the anti-inflammatory agents in an amount sufficient to reduce inflammation of the skin. It should be understood, with reference to dermatological conditions, that the anti-inflammatory agents facilitate inhibition or suppression of inflammation any where on the skin.

An antioxidant can also be used as the active agent in the methods of the invention. Antioxidants react with free radicals to neutralize the free radicals' effects, thus minimizing damage to cell membranes. The antioxidant is typically a vitamin C source and preferably is ascorbic acid, or a pharmaceutically acceptable salt or ester thereof. More preferably, the antioxidant is ascorbyl palmitate, dipalmitate L-ascorbate, sodium L-ascorbate-2-sulfate, or an ascorbic salt, such as sodium, potassium, or calcium ascorbate, or mixtures thereof. When oral formulations of the active agent are used, it is preferred that a non-acidic form of vitamin C be used to reduce the stomach irritation that may occur when using an acidic form. The vitamin C source is present in a composition in about 5 to 50 weight percent, preferably about 7 to 40 weight percent, and more preferably about 10 to 25 weight percent. A unit dose of this primary vitamin C source is typically about 40 mg to 400 mg, preferably about 60 mg to 300 mg, and more preferably about 80 to 150 mg. Vitamin C is also approved by the FDA and has wide consumer acceptance, so that it can be used in amounts as high as 10,000 mg, if desired.

Amino acids can also be used as an active agent. Amino acids are sub-units of protein and aid in building cells, including skin cells. Preferably two or more amino acids are used in combination. Either the L- or D-forms of amino acids are acceptable. Lysine and proline are the most preferred amino acids and are advantageously used in combination. Cysteine, methionine or other amino acids can also be used, if desired. The amino acids may be included in a soluble form such as the hydrochloride salt, i.e., L-Lysine hydrochloride. The amino acids are each present in the pharmaceutical compositions useful in the methods of the invention in an amount ranging from about 2 to 25 weight percent each, preferably about 4 to 20 weight percent each, and more preferably about 6 to 15 weight percent of the pharmaceutical composition. A unit dose for each amino acid is typically about 35 mg to 200 mg each, preferably about 50 mg to 150 mg each, and more preferably about 70 mg to 120 mg. Additional useful forms of amino acid can include a cysteine source, preferably N-acetyl cysteine, which can be present in an amount ranging from about 1 to 10 weight percent, preferably about 2 to 8 weight percent, and more preferably about 3 to 6 weight percent of the pharmaceutical composition. A methionine source, preferably L-selenomethionine, can be present in an amount ranging from about 0.1 to 5 weight percent, preferably 0.2 to 3 weight percent, and more preferably 0.3 to 1 weight percent of the composition, wherein the selenium component is between about 0.1 to 3 weight percent of the methionine source.

One or more transition metal compounds can also be used as an active agent useful in the methods of the invention. The transition metals are typically included in an amount effective to bind collagen and elastic tissue to rebuild the skin. Certain transition metal compounds inhibit the elastase enzyme to inhibit collagen and elastic tissue breakdown. Preferred transition metals include zinc, manganese and copper, with combinations thereof being most preferred.

The zinc component may be any zinc compound or pharmaceutically acceptable salt thereof, but more preferably is zinc complexed with an amino acid, and most preferably is zinc monomethionine, wherein the zinc is typically present in an amount ranging from about 10 to 30 weight percent of the complex. The zinc component is present in an amount ranging from about 1 to 10 weight percent, more preferably about 2 to 7 weight percent, and most preferably about 3 to 5 weight percent of the pharmaceutical composition.

The manganese component may be any manganese compound or pharmaceutically acceptable salt thereof, but more preferably is a manganese component which is at least partially complexed with a vitamin C source, and most preferably is manganese ascorbate or manganese ascorbic acid, wherein the manganese is typically present in an amount ranging from about 5 to 20 weight percent of the complex. When complexed with vitamin C, this vitamin C source may be included in the overall percentage of vitamin C in the pharmaceutical composition. The manganese component is present in an amount ranging from about 1 to 10 weight percent, more preferably about 2 to 7 weight percent, and most preferably about 2.5 to 4 weight percent of the pharmaceutical composition.

The copper component can be any copper compound or pharmaceutically acceptable salt thereof, but preferably is copper sebacate, wherein the copper is typically present in an amount ranging from about 5 to 20 weight percent of the copper sebacate. The copper component is present in an amount ranging from about 0.1 to 5 weight percent, preferably about 0.2 to 3 weight percent, and more preferably about 0.3 to 1 weight percent of the pharmaceutical composition.

A catechin-based compound can also be used as an active agent in the methods of the invention. The catechin-based preparation, similar to vitamin C, inhibits elastase and collagenase, which is another enzyme that attacks elastic tissue and collagen. The catechin-based preparation is preferably a proanthanol or proanthocyanidin, more preferably a proanthocyanidin, and most preferably grape seed extract. These compounds are considered to be secondary antioxidants, because they are present in lesser amounts than the primary antioxidant. The catechin-based preparation can be present in an amount ranging from about 0.5 to 5 weight percent, more preferably about 0.6 to 3 weight percent, and most preferably about 0.7 to 2 weight percent of the pharmaceutical composition.

Chondroitin or a pharmaceutically acceptable salt or ester thereof can be present in an amount ranging from about 3 to 17 weight percent, preferably about 4 to 12 weight percent each, and more preferably about 5 to 8 weight percent each of the pharmaceutical composition. The chondroitin component preferably is present as a sulfate or succinate, and more preferably is chondroitin sulfate, wherein the chondroitin is preferably present as about 65 to 95 weight percent of the salt.

Other active agents useful in the methods of the invention include, but are not limited to, a vitamin E source, a vitamin B₃ source, quercetin powder, pyridoxal 5 phosphate-Co B₆, and a vitamin A source.

The vitamin E source preferably is a sulfate or succinate vitamin E complex, and more preferably is D-alpha tocopheryl acid succinate. The vitamin E source is present in an amount ranging from about 1 to 15 weight percent, preferably about 2 to 12 weight percent, and more preferably about 3 to 10 weight percent of the pharmaceutical composition. In any event, no more than 1,500 IU should be ingested per day, as Vitamin E becomes toxic at higher doses.

The vitamin B₃ source preferably is niacinamide, and the source is present in an amount ranging from about 0.5 to 15 weight percent, preferably about 1 to 12 weight percent, and more preferably about 1.5 to 10 weight percent of the pharmaceutical composition.

The vitamin A source preferably is retinyl palmitate or other retinyl esters, retinoic acid, or Retinol. The Retinol facilitates normal skin production, particularly epidermal normalization, and, when used, is typically present in an amount ranging from about 0.01 to 6 weight percent, preferably about 0.1 to 5 weight percent, more preferably 0.2 to 3 weight percent, and most preferably 0.3 to 1 weight percent of the pharmaceutical composition of the pharmaceutical composition. In a more preferred form, the amount of vitamin A dosage is about 500,000 IU/gram per unit dose. Vitamin A is toxic at high levels, such that no more than 400,000 IU should be cumulatively ingested per day for greater than six months.

Quercetin powder can also be used as an active agent. The quercitin powder is quercetin dihydrate, which is typically present in an amount ranging from about 0.5 to 15 weight percent, preferably about 1 to 12 weight percent, and more preferably about 1.5 to 10 weight percent of the pharmaceutical composition.

The pyridoxal 5 phosphate-Co B6, also known as P-5-P monohydrate, is typically present in an amount ranging from about 0.1 to 5 weight percent, preferably 0.2 to 3 weight percent, and more preferably 0.3 to 1 weight percent of the pharmaceutical composition.

In another embodiment, the active agent used in the methods of the invention is a pharmaceutically acceptable antimicrobial agent. Any pharmaceutically acceptable antimicrobial agent available to those of ordinary skill in the art may be used, but preferably at least one of an antibacterial agent, antifungal agent, antiviral agent, or anthelmintic will be used in the methods of the invention. A single broad spectrum antimicrobial agent, i.e., one that is believed to have at least two of antibacterial, antifungal, and antiviral efficacy, include: echinacea, golden seal, benzalkonium chloride, benzethonium chloride, iodine, grape seed extract, pomegranate extract, green tea extract or polyphenols, and the like, or combinations thereof, may be used in the methods of the invention. Another suitable antimicrobial agent includes the class of anthelmintics, such as metronidazole, to facilitate treatment of, e.g., tricomona infection. Preferred antiviral agents include, but are not limited to, acyclovir, tamvir, penciclovir, and the like, and mixtures thereof. Preferred antibacterial agents include, but are not limited to, triclosan, neomycin, polymyxin, bacitracin, clindamycin, benzoyl peroxide, a tetracycline, a sulfa drug, a penicillin, a quinolone, a cephalosporin, and mixtures thereof. Preferred antifungal agents include, but are not limited to, famesol, econazole, fluconazole, clotrimazole, ketoconazole, calcium or zinc undecylenate, undecylenic acid, butenafine hydrochloride, ciclopirox olaimine, miconazole nitrate, nystatin, sulconazole, terbinafine hydrochloride, and the like, and mixtures thereof. Exemplary tetracyclines include doxycycline and minocycline. An exemplary sulfa drug includes sulfacetamde. An exemplary cephalosporin includes cephalexin (commercially available as KEFLEX). Exemplary quinolones include the floxacins, such as loemfloxacin, of loxacin, and trovafloxacin. It should be readily understood that any salts, isomers, pro-drugs, metabolites, or other derivatives of these antimicrobial agents may also be included as the antimicrobial agent in accordance with the invention. The antimicrobial agent is typically present in an amount ranging from about 0.01 to 1.5 weight percent, preferably from about 0.1 to 1.2 weight percent, and more preferably from about 0.3 to 1 weight percent of the pharmaceutical composition. The antimicrobial agent inhibits the formation, and may further reduce, the presence of microbes that cause redness, inflammation, and irritation of the skin.

The active agent used in the methods of the invention can be an immuno-modulator to stimulate or suppress the bodies immune system. The immuno-modulator can be an immuno-enhancer or an immumo-suppressant. A suitable immuno-enhancer useful in the method of the invention is Aldara (Immiquimod). Immuno-enhancers can be useful in treating, for example, warts or pre-cancerous lesions. The immuno-enhancer may be present in the pharmaceutical compositions used in the methods of the invention in an amount from about 0.1 to 10 weight percent, preferably from about 0.5 to 5 by weight percent of the pharmaceutical composition. Suitable immuno-suppressants useful in the methods of the invention include Tacromilus or Pimercolimus. Immuno-suppressants can be useful in treating, for example, eczema. The immuno-suppressant may be present in the pharmaceutical compositions used in the methods of the invention in an amount from about 0.1 to 10 weight percent, preferably from about 0.5 to 5 by weight percent of the pharmaceutical composition.

In one embodiment, the pharmaceutical composition further comprises an exfoliant to help remove dead or dying skin cells and further improve the skin's own ability to absorb moisture directly from the atmosphere in combination with one or more hydrophilic agents to help absorb moisture from the atmosphere and hydrate the skin or in combination with one or more a hydrophobic agents to inhibit or prevent moisture loss by the skin. More preferably, the active agent is one or more of a hydrophilic agent and a hydrophobic agent in combination with an exfoliant. It is believed that the combination of an exfoliant, a hydrophilic moisturizer, and a hydrophobic moisturizer has an unexpected synergistic effect that helps other active agents penetrate the skin. The exfoliant functions by removing dead or dying skin cells, enabling the skin to better absorb moisture from the atmosphere, the hydrophobic agents prevent the loss of water from the skin, and the hydrophilic agents moisturize the skin by absorbing moisture or facilitating hydration of the skin.

The exfoliant may be an enzymatic exfoliant, or an acidic exfoliant. Any enzymatic exfoliant known to those skilled in the art may be used in the methods of the invention. Examples of enzymatic exfoliants useful in the methods of the invention include, but are not limited to, papain, from papaya, and bromalein, from pineapple.

Examples of acidic exfoliants include, but are not limited to a mono- or poly-hydroxy acid, tannic acid, or a mixture thereof, or a pharmaceutically acceptable salt or ester thereof. One of ordinary skill in the art will readily be able to select and prepare suitable mono- or poly-hydroxy acids for use in the methods of the invention, for example, alkyl hydroxycarboxylic acids, aralkyl and aryl hydroxycarboxylic acids, polyhydroxy-carboxylic acids, and hydroxy-polycarboxylic acids. One of ordinary skill in the art would typically select one or more of the following mono- or poly-hydroxy acids: 2-hydroxyacetic acid (glycolic acid); 2-hydroxypropanoic acid (lactic acid); 2-methyl 2-hydroxypropanoic acid; 2-hydroxybutanoic acid; phenyl 2-hydroxyacetic acid; phenyl 2-methyl 2-hydroxyacetic acid; 3-phenyl 2-hydroxyacetic acid; 2,3-dihydroxypropanoic acid; 2,3,4-trihydroxybutanoic acid; 2,3,4,5,6-pentahydroxyhexanoic acid; 2-hydroxydodecanoic acid; 2,3,4,5-tetrahydroxypentanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy-3-methylpentanoic acid; 4-hydroxydecanoic acid; 5-hydroxydecanoic acid; aleuritic acid; 2-hydroxypropanedioic acid; 2-hydroxybutanedioic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid; citric acid, isocitric acid, agaricic acid, quinic acid, glucoronic acid, glucoronolactone, galactoronic acid, galactoronolactone, uronic acids, uronolactones, ascorbic acid, dihydroascorbic acid, dihydroxytartaric acid, tropic acid, ribonolactone, gluconolactone, galactonolactone, gulonolactone, mannonolactone, citramalic acid; pyruvic acid, hydroxypyruvic acid, hydroxypyruvic acid phosphate and esters thereof; methylpyruvate, ethyl pyruvate, propyl pyruvate, isopropyl pyruvate; phenyl pyruvic acid and esters thereof; methyl phenyl pyruvate, ethyl phenyl pyruvate, propyl phenyl pyruvate; formyl formic acid and esters thereof; methyl formyl formate, ethyl formyl formate, propyl formyl formate; benzoyl formic acid and esters thereof; methyl benzoyl formate, ethyl benzoyl formate and propyl benzoyl formate; 4-hydroxybenzoyl formic acid and esters thereof; 4-hydroxyphenyl pyruvic acid and esters thereof; and 2-hydroxyphenyl pyruvic acid and esters thereof.

It should be understood that one or more derivatives of the above poly-hydroxy acidic component, such as esters or lactones thereof, are also suitably used. One of ordinary skill in the art will also understand that various hydroxy acids described in U.S. Pat. Nos. 5,547,988 and 5,422,370 are also suitable for use in the methods of the invention. The acidic component is present in the composition and methods in an amount sufficient to exfoliate, i.e., remove dead or dying skin cells, from at least a portion of the skin. The acidic component is typically present in an amount ranging from about 0.1 to 12 weight percent, preferably about 1 to 11 weight percent, more preferably from about 4 to 10 weight percent of the composition. For example, the poly-hydroxy acidic component may be from about 0.1 to about 3 weight percent citric acid in combination with up to about 2 weight percent salicylic acid. Exfoliants are typically used in topical compositions.

The term “pharmaceutically acceptable salt” refers to a salt prepared from pharmaceutically acceptable non-toxic acid. Examples of suitable inorganic metallic bases for salts formation with the acid compounds of the invention include, but are not limited to, aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), and procaine.

In one embodiment, the pharmaceutical composition useful in the methods of the invention further comprises hydrogen peroxide. Hydrogen peroxide, if present as an active agent, is present in an amount sufficient to cleanse at least a portion of the skin. “Cleanse” as used herein includes the removal of dirt, debris, air pollutants, desquamating cells, and cutaneous secretions of the skin. Preferably, the hydrogen peroxide is used in an amount to cleanse the skin without substantial irritation. The hydrogen peroxide is typically present in an amount ranging from about 0.01 to 6 weight percent, preferably 0.05 to 4 weight percent, and more preferably 0.1 to 1 weight percent of the pharmaceutical composition. Without wishing to be bound by theory, it is believed that hydrogen peroxide assists in improving penetration into the skin of other active agents used in the methods of the invention. Hydrogen peroxide is typically used in topical compositions.

In one embodiment, the pharmaceutical composition used in the methods of the invention includes hydrogen peroxide, one or more moisturizing agents, and at least one other active agent. Without wishing to be bound by theory it is believed that hydrogen peroxide, one or more moisturizing agents, and other active agents useful in the methods of the invention interact in a synergistic manner to increase the intracellular water content. Together, the hydrogen peroxide and one or more moisturizing agents cleanse the skin, remove substances foreign to the skin, and moisturize the skin to improve penetration of the other active agents to increase the intracellular water content of the patient.

The pharmaceutical compositions containing the active agent used in the methods of the invention may further include one or more excipients such as surfactants, stabilizers, preservatives, coloring agents, anti-oxidants, water, buffering agents, emulsifying agents, thickeners, solvents, perfuming agents, and the like. Preferably, the water is deionized water. It should be understood that water includes the remainder of a given composition after other ingredients are determined. Any pharmaceutically acceptable surfactant, stabilizer, preservative, coloring agent, buffering agent, emulsifying agent, thickener, solvent, or perfirming agent may be used. Representative compounds or mixtures are discussed below.

Representative surfactants, including both the foaming and non-foaming type, including, but not limited to, sodium laureth sulfate, sodium laureth-13 carboxylate, disodium laureth sulfosuccinate, disodium cocoamphodiacetate, and the like, and mixtures thereof. More preferably, at least one amphoteric surfactant is included in the composition, such as disodium cocoamphodiacetate. The amphoteric surfactant, in combination with citric acid, inhibits hydrogen peroxide decomposition. The surfactant component may be present in an amount from about 10 to 90 weight percent, preferably about 20 to 80, and more preferably about 30 to 70 weight percent of the pharmaceutical composition.

A representative stabilizer that can be used in the pharmaceutical compositions used in the methods of the invention includes glycol stearate or PEG-150 distearate. The stabilizer, when used, is typically present in an amount from about 0.1 to 5 weight percent of the pharmaceutical composition.

Representative preservatives that can be used in the pharmaceutical compositions used in the methods of the invention include tetrasodium ethylene-diamine tetraacetic acid (EDTA), methylparaben, benzophenone-4, methylchloroisothiazolinone, methylisothiazolinone, and the like, and mixtures thereof. Preservatives, when used, are typically present in an amount from about 0.01 to 6 weight percent, preferably about 0.05 to 4 weight percent, and more preferably from about 0.1 to 2 weight percent of the pharmaceutical composition.

Representative coloring agents used in the pharmaceutical compositions used in the methods of the invention include FD&C Green No. 3, Ext. D&C Violet No. 2, FD&C Yellow No. 5, FD&C Red No. 40, and the like, and mixtures thereof. The coloring agents, when used, are typically present in an amount from about 0.001 to 0.1 weight percent, and preferably from about 0.005 to 0.05 weight percent of the pharmaceutical composition.

The pharmaceutical compositions used in the methods of the invention may also include one or more of a local analgesic or anesthetic, anti-yeast agent, antiperspirant, anti-psoriatic agent anti-aging agent, anti-wrinkle agent, sun screen and sun blocking agent, skin lightening agent, depigmenting agent, vitamin, hormone and retinoid. Particularly preferred are agents further comprising a local analgesic or anesthetic to alleviate the pain and discomfort associated with inflammatory skin diseases. Local anesthetic include, but are not limited to, lidocaine.

The active agents can be administered orally, topically, by a mixture of oral and topical doses, or through any other suitable route of administration. Other suitable routes include, for example, rectal, parenteral, intravenous, transdermal, subcutaneous, and intramuscular. One of ordinary skill in the art would know or could determine without undue experimentation the appropriate oral and topical doses of the active agents used in the methods of the invention. In one embodiment, the active agent is administered orally. In another embodiment, the active agent is administered orally. One of ordinary skill in the art would readily know what active agents can be administered topically and what active agents can be administered orally. In another embodiment, moisturizing agents, exfoliants, and hydrogen peroxide are administered topically.

The magnitude of a prophylactic or therapeutic dose of the active agent used in the methods of the invention will vary with the severity of the condition to be treated. The dose, and perhaps the dose frequency, will also vary according to the age, body weight, and response of the individual patient. In general, a preferred topical daily dose range, in single or divided doses, for the conditions described herein should be from about 1 mg to 20,000 mg, more preferably about 2,000 mg to 16,000 mg, and most preferably about 6,000 mg to 10,000 mg of the active components (i.e., excluding excipients and carriers).

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know how and when to interrupt, adjust, or terminate therapy in conjunction with individual patient response.

Those of ordinary skill in the art will also understand that topical effectiveness of active agents requires percutaneous absorption and bioavailability to the target site. Thus, the compositions and methods of the invention require penetration through the stratum corneum into the epidermal layers, as well as sufficient distribution to the sites targeted for pharmacologic action.

Suitable dosage forms for topical administration include, but are not limited to, dispersions, lotions; creams; gels; pastes; powders; aerosol sprays; syrups or ointments on sponges or cotton applicators; and solutions or suspensions in an aqueous liquid, non-aqueous liquid, oil-in-water emulsion, or water-in-oil liquid emulsion. Because of its ease of administration, a cream, lotion, or ointment represents the most advantageous topical dosage unit form, in which case liquid pharmaceutical carriers may be employed in the composition. These creams, lotions, or ointments, may be prepared as rinse-off or leave-on products, as well as two stage treatment products for use with other skin cleansing or managing compositions. In a preferred embodiment, the compositions are administered as a rinse-off product in a higher concentration form, such as a gel, and then a leave-on product in a lower concentration to avoid irritation of the skin. Each of these forms is well understood by those of ordinary skill in the art, such that dosages may be easily prepared to incorporate the pharmaceutical composition of the invention.

Desirably, each unit dose, e.g., gel, cream, or ointment, contains from about 1 mg to 2,000 mg of the active ingredients, preferably about 200 mg to 1,600 mg, and more preferably about 600 mg to 1,000 mg of the composition.

Suitable oral dosage forms include tablets, troches, dispersions, suspensions, solutions, and capsules. Desirably, each unit dose administered orally contains from about 1 mg to 2,000 mg of the active ingredients, preferably about 20 mg to 1,600 mg, and more preferably about 50 mg to 1,000 mg of the composition.

The methods of the invention may further comprise administering one or more additional active agents by a route of administration other than orally or topically. Any suitable route of administration may be employed for providing the patient with an effective dosage of the additional component including, but not limited to, intraoral, rectal, parenteral, topical, epicutaneous, transdermal, subcutaneous, intramuscular, intranasal, sublingual, buccal, intradural, intraocular, intrarespiratory, or nasal inhalation and like forms of administration.

The pharmaceutical compositions used in the methods of the invention may be prepared by any of the methods of pharmacy, but all methods include the step of bringing into association the carrier(s) with the active ingredient, which constitutes one or more necessary ingredients. In general, the compositions are prepared by uniformly and intimately admixing the active ingredient with liquid carriers or finely divided solid carriers or both, and then, if necessary, shaping the product into the desired presentation.

The methods of the invention typically include administering the active agent to the patient daily for about 1 to about 50 days prior to re-measuring the intracellular water content. In one embodiment, the active agent is administered to the patient for at least about 7 days prior to re-measuring the intracellular water content. In one embodiment, the active agent is administered to the patient daily for at least about 14 days prior to re-measuring the intracellular water content. In one embodiment, the active agent is administered to the patient daily for at least about 28 days prior to re-measuring the intracellular water content. In one embodiment, the active agent is administered to the patient daily for at least about 35 days prior to re-measuring the intracellular water content. In one embodiment, the active agent is administered to the patient daily for at least about 42 days prior to re-measuring the intracellular water content.

Typically, the methods of the invention result in the post-treatment intracellular water content measurement, measured as a percent, being about 0.01 to about 10 percentage units greater than the initial intracellular water content, measured as a percent. In one embodiment, the post-treatment intracellular water content measurement, measured as a percent, is about 0.25 to about 5 percentage units greater than the initial intracellular water content, measured as a percent. In one embodiment, the post-treatment intracellular water content measurement, measured as a percent, is about 0.75 to about 3 percentage units greater than the initial intracellular water content, measured as a percent. In one embodiment, the post-treatment intracellular water content measurement, measured as a percent, is at least 0.5 percentage units higher than the initial intracellular water content measurement. In one embodiment, the post-treatment intracellular water content measurement, measured as a percent, is at least 1 percentage units higher than the initial intracellular water content measurement. In one embodiment, the post-treatment intracellular water content measurement, measured as a percent, is at least 3 percentage units higher than the initial intracellular water content measurement. In one embodiment, the post-treatment intracellular water content measurement, measured as a percent, is at least 5 percentage units higher than the initial intracellular water content measurement.

Typically, the methods of the invention result in the post-treatment extracellular water content measurement, measured as a percent, being about 0.01 to about 10 percentage units less than the initial extracellular water content, measured as a percent. In one embodiment, the post-treatment extracellular water content measurement, measured as a percent, is about 0.25 to about 10 percentage units less than the initial extracellular water content, measured as a percent. In one embodiment, the post-treatment extracellular water content measurement, measured as a percent, is about 0.75 to about 5 percentage units less than the initial extracellular water content, measured as a percent. In one embodiment, the post-treatment extracellular water content measurement, measured as a percent, is at least about 0.5 percentage units less than the initial extracellular water content measurement. In one embodiment, the post-treatment extracellular water content measurement, measured as a percent, is at least about 1 percentage units less than the initial extracellular water content measurement. In one embodiment, the post-treatment extracellular water content measurement, measured as a percent, is at least about 3 percentage units less than the initial extracellular water content measurement. In one embodiment, the post-treatment extracellular water content measurement, measured as a percent, is at least about 5 percentage units less than the initial extracellular water content measurement.

Typically, the methods of the invention result in post-treatment basal metabolic rates, measured in calories, being about 1 to about 300 calories higher than the initial basal metabolic rate. In one embodiment, the post-treatment basal metabolic rate is about 5 to about 100 calories higher than the initial basal metabolic rate. In one embodiment, the post-treatment basal metabolic rate is about 10 to about 50 calories higher than the initial basal metabolic rate. In one embodiment, the post-treatment basal metabolic rate is at least about 5 calories higher than the initial basal metabolic rate. In one embodiment, the post-treatment basal metabolic rate is at least about 25 calories higher than the initial basal metabolic rate. In one embodiment, the post-treatment basal metabolic rate is at least about 100 calories higher than the initial basal metabolic rate. In one embodiment, the basal metabolic rate increase, measured as a percentage of the initial basal metabolic rate, is from about 1% to about 15%. In one embodiment, the basal metabolic rate increase, measured as a percentage of the initial basal metabolic rate, is from about 3% to about 12%. In one embodiment, the basal metabolic rate increase, measured as a percentage of the initial basal metabolic rate, is from about 5% to about 10%.

In addition to the measurement of intracellular water content, the methods of the invention can incorporate the use of additional feedback, in the form of data or observations, to correlate the increase in intracellular water content of the skin with healthier and more youthful appearing skin. Such additional feedback can include, but is not limited to, photographic imagery, black light or UV imagery, ultrasound measurements, chemical testing within the skin, surface moisture measurements, a count of the number of acne lesions on the skin, and computerized scans of the skin.

In one embodiment, the method further comprises taking a first photographic image of the patient's skin before obtaining the initial intracellular water content measurement and a second photographic image of the patient's skin after the post-treatment intracellular water content measurement and comparing the first photographic image and the second photographic image to correlate the appearance of the skin or the reduction in the dermatological condition with the increase in intracellular water content of the skin and to assess whether a further treatment is needed to further improve the dermatological condition. For example, a Canon-DS6041 Macro Twin Light camera (commercially available from Canon, Inc., Tokyo, Japan or Canon U.S.A., Inc., Lake Success, N.Y.) can be used to take a photographic image of the patient's skin before obtaining the initial intracellular water content measurement and after obtaining the post-treatment intracellular water content measurement.

Photographs of subjects can also be taken using a Canfield Clinical System (commercially available from Canfield of Fairfield, N.J.). This system allows the dermatologist or cosmetologist to precisely and reproducibly position the head of a subject and to carefully control the lighting, film type and processing so that the comparison of photographs before treatment and after treatment with the active agent can accurately represent changes resulting from the treatment.

Changes in skin hydration, i.e., total water content in the stratum corneum, the outer layer of the skin, can also be measured using a CORNEOMETER (CM-820, commercially available from Courage and Khazaka of Germany) that measures changes in the capacitance of the skin resulting from small changes in the degree of hydration. The CORNEOMETER expresses the capacitance of the skin in arbitrary unit of skin hydration (H). The instrument is capable of measuring the moisture of the stratum corneum to a depth of 0.1 mm and is used to measure the effects of cosmetic preparations on the moisture content of the skin. Tests using the CORNEOMETER are typically conducted by taking 3 measurements on the face, one at each of the right cheek and left cheek and one at the center of the skin, for each patient. The three measurements are then averaged for each patient.

In another embodiment, skin layer thickness measurements are measured using ultrasound technology. These measurements are taken both before measuring the patient's initial intracellular water content and after measuring the post-treatment intracellular water content.

### EXAMPLES

The invention is further defined by reference to the following example. The example is representative, and should not be construed to limit the scope of the invention.

### Example 1

### Case Study

The intracellular water content of a patient is measured using a bioelectrical impedance analyzer such as the Bioelectrical Body Composition Analyzer (“BIA,” commercially available from RJL Systems of Clinton Township, Michigan). The instrument can measure a person's intracellular water content, total body water content, extracellular water content, body fat, and lean body mass.

In one case study, a 32 year old woman suffering from rosacea, slight acne, and generally dull skin was treated according to the method of the present invention. As a first step the dermatologist determined the percent intracellular water content, percent extracellular water content, percent total body water content, and percent body fat of the patient using the Bioelectrical Body Composition Analyzer. The following initial measurements were obtained:

| BIA Results | Actual |
|---|---|
| % Body fat | 39.7 |
| % Total body water content | 46.3 |
| % Intracellular water content | 24.9 |
| % Extracellular water content | 21.4 |

On the day the above-described measurements were performed, the patient was instructed to administer the following pharmaceutical compositions according to directions prescribed on the label: (i) Global environmental Shield Essential C Night Moisture & Eye Cream (which contains Water (Aqua), Cetearyl Ethylhexanoate, Ethylhexyl Hydroxystearate, Butylene Glycol, Stearic Acid, Cetearyl Alcohol, Glyceryl Stearate, PEG-100 Stearate, DEA-Cetyl Phosphate, Polysorbate 60, Chitosan Ascorbate, Lysine Lauroyl Methionate, Rice Amino Acids, Zinc Aspartate, Phospholipids, Tocopheryl Acetate, Retinyl Palmitate, Ascorbyl Palmitate, Phytonadione, Ethyl Linoleate, Panthenol, Dimethicone, Ginkgo Biloba Leaf Extract, Vitis Vinifera (Grape) Seed Extract, Passiflora Incarnata Extract, Hydrolyzed Ulva Lactuca Extract, Hydrolyzed Enteromorpha Compressa, Butyrospermum Parkii (Shea Butter), Sodium PCA, Isostearic Acid, Glycerin, Carbomer, Disodium EDTA, Triethanolamine, Phenoxyethanol, Methylparaben, Propylparaben, Benzyl Benzoate, Citronellol, Geraniol, Limonene, Linalool, Citrus Grandis (Grapefruit) Peel Oil, Citrus Aurantium Dulcis (Orange) Oil, Aniba Rosaeodora (Rosewood) Wood Oil, Geranium Maculatum Oil, Lavandula Angustifolia (Lavender) Oil, Thymus Vulgaris (Thyme) Oil, Yellow 6 (CI 15985)); (ii) Environmental Shield Vitamin C Home Infusion Kit, i.e., Environmental Shield Vitamin C Infusion Treatment Gel—Home Kit (which contains Glycerin, Glyceryl Polymethacrylate, Cyanocobalamin, Retinyl Palmitate, Beta-Carotene, Ranunculus Ficaria Extract, Rice Amino Acids, Lysine Lauroyl Methionate, Zinc Aspartate, Chitosan Ascorbate) and Environmental Shield Renewal Complex Professional Gel (containing ascorbic acid and tapioca starch); and (iii) Pomphenol™ Supplement (the contents of which are disclosed below), and (iv) DRC (each commercially available from Murad Inc., El Segundo, Calif.).

After 28 days of treatment, the percent intracellular water content, percent extracellular water content, percent total body water content, and percent body fat of the patient was measured again. The following measurements were obtained:

| BIA Results | Actual |
|---|---|
| % body fat | 38.5 |
| % total body water content | 46.9 |
| % Intracellular water content | 25.5 |
| % Extracellular water content | 21.4 |

After 4 weeks of treatment with active agents, the patient's acne that accompanied her rosacea had completely cleared, and the inflammation of her skin had reduced significantly. This improvement was accompanied by an increase in her intracellular water content of about 1%. Concurrent with re-measuring the percent intracellular water content, percent extracellular water content, percent total body water content, and percent body fat of the patient; photographs were taken of the patient's face using a Canon-DS6041 Macro Twin Light camera to document the patient's progress.

### Example 2

### Case Study

In another case study, a 31 year old woman suffering from pigmentation, sun damage, and very dry skin was treated according to the method of the present invention. As a first step, the clinician determined the percent intracellular water content, percent extracellular water content, percent total body water content, and percent body fat of the patient using the Bioelectrical Body Composition Analyzer. The following initial measurements were obtained:

| BIA Results | Actual |
|---|---|
| % Body fat | 36.0 |
| % Total body water content | 48.1 |
| % Intracellular water content | 27.6 |
| % Extracellular water content | 20.5 |

On the day the above-described measurements were performed, the patient was instructed to administer the following pharmaceutical compositions according to directions prescribed on the label: Pure Skin® Supplement and Wet Suit™ Supplement (each commercially available from Murad Inc., El Segundo, Calif. and the contents of which are disclosed below).

After 68 days of treatment, the percent intracellular water content, percent extracellular water content, percent total body water content, and percent body fat of the patient was measured again. The following measurements were obtained:

| BIA Results | Actual |
|---|---|
| % body fat | 32.4 |
| % total body water content | 50.0 |
| % Intracellular water content | 30.3 |
| % Extracellular water content | 19.7 |

After 68 days of treatment the patient's skin was firmer, more hydrated, showed less sun-related damage and the wrinkles around her eyes had decreased. This improvement was accompanied by an increase in her intracellular water content of about 2.7%.

### Example 3

### Case Study

In another case study, a 35 year old woman suffering from eczema on her hands, elbows, legs and face was treated according to the method of the present invention. As a first step, the clinician determined the percent intracellular water content, percent extracellular water content, percent total body water content, and percent body fat of the patient using the Bioelectrical Body Composition Analyzer. The following initial measurements were obtained:

| BIA Results | Actual |
|---|---|
| % Body fat | 60.3 |
| % Total body water content | 33.7 |
| % Intracellular water content | 19.1 |
| % Extracellular water content | 14.6 |

On the day the above-described measurements were performed, the patient was instructed to administer the following pharmaceutical compositions according to directions prescribed on the label: Pure Skin® Supplement and Wet Suit™ Supplement (each commercially available from Murad Inc., El Segundo, Calif. and the contents of which are disclosed below).

After 30 days of treatment, the percent intracellular water content, percent extracellular water content, percent total body water content, and percent body fat of the patient was measured again. The following measurements were obtained:

| BIA Results | Actual |
|---|---|
| % body fat | 58.4 |
| % total body water content | 35.0 |
| % Intracellular water content | 22.2 |
| % Extracellular water content | 12.8 |

After 30 days of treatment the patient's skin was more hydrated, and she exhibited noticeable improvement on her hands, elbows and face. This improvement was accompanied by an increase in her intracellular water content of about 3.1%.

### Example 4

### Case Study

In another case study, a 70 year old woman who had badly broken capillaries (spider veins) and easily bruised skin was treated according to the method of the present invention. As a first step, the clinician determined the percent intracellular water content, percent extracellular water content, percent total body water content, and percent body fat of the patient using the Bioelectrical Body Composition Analyzer. The following initial measurements were obtained:

| BIA Results | Actual |
|---|---|
| % Body fat | 29.9 |
| % Total body water content | 52.1 |
| % Intracellular water content | 26.7 |
| % Extracellular water content | 25.4 |

On the day the above-described measurements were performed, the patient was instructed to administer the following pharmaceutical compositions according to directions prescribed on the label: Youth Builder® Supplement and Wet Suit™ Supplement (each commercially available from Murad Inc., El Segundo, Calif. and the contents of which are disclosed below).

After 30 days of treatment, the percent intracellular water content, percent extracellular water content, percent total body water content, and percent body fat of the patient was measured again. The following measurements were obtained:

| BIA Results | Actual |
|---|---|
| % body fat | 28.1 |
| % total body water content | 53.5 |
| % Intracellular water content | 28.0 |
| % Extracellular water content | 25.5 |

After 30 days of treatment the patient's skin had thickened and she had less bruising. This improvement was accompanied by an increase in her intracellular water content of about 1.3%.

### Example 5

### Case Study

In another case study, a 70 year old man who was diabetic and had high blood pressure was treated according to the method of the present invention. As a first step, the clinician determined the percent intracellular water content, percent extracellular water content, percent total body water content, and percent body fat of the patient using the Bioelectrical Body Composition Analyzer. The following initial measurements were obtained:

| BIA Results | Actual |
|---|---|
| % Body fat | 21.1 |
| % Total body water content | 57.1 |
| % Intracellular water content | 32.6 |
| % Extracellular water content | 24.5 |

On the day the above-described measurements were performed, the patient was instructed to administer the following pharmaceutical compositions according to directions prescribed on the label: Firm and Tone, Wet Suit™ Supplement, and Pomphenol™ Supplement (each commercially available from Murad Inc., El Segundo, Calif. and the contents of which are disclosed below).

After 26 days of treatment, the percent intracellular water content, percent extracellular water content, percent total body water content, and percent body fat of the patient was measured again. The following measurements were obtained:

| BIA Results | Actual |
|---|---|
| % body fat | 19.7 |
| % total body water content | 58.6 |
| % Intracellular water content | 33.8 |
| % Extracellular water content | 24.8 |

After 26 days of treatment the patient's blood pressure had lowered and he felt more energetic. This improvement was accompanied by an increase in his intracellular water content of about 1.2%.

### Example 6

### Case Study

In another case study, a 17 year old female who had a bad case of acne was treated according to the method of the present invention. As a first step, the clinician determined the percent intracellular water content, percent extracellular water content, percent total body water content, and percent body fat of the patient using the Bioelectrical Body Composition Analyzer. The following initial measurements were obtained:

| BIA Results | Actual |
|---|---|
| % Body fat | 31.1 |
| % Total body water content | 52.4 |
| % Intracellular water content | 27.5 |
| % Extracellular water content | 24.9 |

On the day the above-described measurements were performed, the patient was instructed to administer the following pharmaceutical compositions according to directions prescribed on the label: (i) Pure Skin® (the contents of which are disclosed below); (ii) Wet Suit™ (the contents of which are disclosed below); (iii) Exfoliating Acne Treatment Gel (containing salicylic acid (1.0%), Water (Aqua), Alcohol Denatured, Glycolic Acid, Butylene Glycol, Aloe Barbadensis Leaf Extract, Methyl Gluceth-10, PPG-5-Ceteth-20, Hydrogen Peroxide, Rice Amino Acids, Lysine Lauroyl Methionate, Zinc Aspartate, Chitosan Ascorbate, Retinol, Polysorbate 20, Phytonadione, Arnica Montana Flower Extract, Epilobium Angustifolium Extract, Citrus Aurantium Dulcis (Orange) Fruit Extract, Triclosan, Lecithin, Tocopherol, Magnesium Ascorbyl Phosphate, Dipotassium Glycyrrhizate, Palmitoyl Hydroxypropyltrimonium Amylopectin/Glycerin Crosspolymer, Vitis Vinifera (Grape) Seed Extract, Camellia Sinensis Leaf Extract, Hamamelis Virginiana (Witch Hazel) Water, Zinc Acetate, Linoleic Acid, Sodium Hydroxide, Hydroxyethylcellulose, Tetrasodium EDTA); and (iv) Cellular Serum (containing Water (Aqua), Glycerin, Methyl Gluceth-20, Glycolipids, Hyaluronic Acid, Anthemis Nobilis Flower Extract, Lecithin, Tocopherol, Magnesium Ascorbyl Phosphate, Algae Extract, Palmitoyl Hydroxypropyltrimonium Amylopectin/Glycerin Crosspolymer, Vitis Vinifera (Grape) Seed Extract, Yeast Extract, Panax Ginseng Root Extract, Camellia Oleifera Leaf Extract, Malva Sylvestris (Mallow) Extract, Hedera Helix (Ivy) Extract, Cucumis Sativus (Cucumber) Fruit Extract, Sambucus Nigra Flower Extract, Arnica Montana Flower Extract, Parietaria Officinalis Extract, PEG-12 Dimethicone, Propylene Glycol, Hydroxyethylcellulose, Tetrasodium EDTA, Diazolidinyl Urea, Methylparaben, Blue 1 (CI 42090), Yellow 6 (CI 15985)) (each commercially available from Murad Inc., El Segundo, Calif.).

After 18 days of treatment, the percent intracellular water content, percent extracellular water content, percent total body water content, and percent body fat of the patient was measured again. The following measurements were obtained:

| BIA Results | Actual |
|---|---|
| % body fat | 30.3 |
| % total body water content | 53.3 |
| % Intracellular water content | 27.9 |
| % Extracellular water content | 25.4 |

After 18 days of treatment the patient's acne cleared up and she felt like she had more endurance. This improvement was accompanied by an increase in her intracellular water content of about 0.4%.

### Example 7

### Case Studies

The following table presents data from a series of case studies in which the change in a patient's intracellular water content throughout the course of treatment was monitored. The data presented include the age of the patient, the increase in intracellular water content, and relevant clinical observations. The patients were administered a combination of one or more of the following supplements, the active ingredients of which are provided below: Firm and Tone Supplement, Wet Suit™ Cell Hydrating Supplement, Pure Skin® Clarifying Supplement, Youth Builder® Supplement, Pomphenol™ Sunguard Supplement, AM Pack, PM Pack (each commercially available from Murad Inc., El Segundo, Calif.).

Firm and Tone Supplement

| | Amount Per Serving (mg, | |
|---|---|---|
| Ingredient | unless otherwise specified) | |
| Vitamin A Acetate | 4000 | IU |
| Vitamin B-3 (niacinamide) | 60 | |
| Vitamin B-6 | 20 | |
| Vitamin C (ascorbic acid) | 120 | |
| Vitamin E (succinate) | 60 | IU |
| Chromium (from chromium picolinate | 16 | mcg |
| 200 mcg) | | |
| Zinc (amino acid chelate) | 18 | |
| Copper (amino acid chelate) | 800 | mcg |
| Selenium (chelate) | 50 | mcg |
| Garcinia Cabogia (50% HCA) | 250 | |
| Essential Fatty Acids | 190 | |
| Inositol | 100 | |
| L-Lysine | 125 | |
| N-Acetyl Glucosamine | 100 | |
| L-Proline | 100 | |
| Cayenne (40,000 scaville units) | 85 | |
| L-Glycine | 75 | |
| Choline (bitartrate) | 50 | |
| N-Acetyl Cysteine | 50 | |
| Glucosamine Sulfate HCL | 1200 | |
| Curcumin (tumeric) | 40 | |
| Quercetin | 20 | |
| Grape Seed Extract (proanthodyn) | 15 | |
| Phosphatidylcholine (from lecithin) | 2500 | mcg |

Wet Suit Cell Hydrating Supplement

| | Amount Per Serving (mg, | |
|---|---|---|
| Ingredient | unless otherwise specified) | |
| Vitamin C | 120 | |
| Vitamin E (acetate) | 100 | |
| Zinc | 10 | |
| Manganese | 4 | |
| Copper | 2 | |
| Selenium | 150 | mcg |
| Type II Collagen | 250 | |
| Glucosamine Sulfate | 170 | |
| Essential Fatty Acid Complex | 150 | |
| Dipotassium Phosphate | 150 | |
| Choline | 100 | |
| L-Lysine | 90 | |
| L-Glycine | 90 | |
| Aloe Vera Concentrate | 60 | |
| Potassium Sulfate | 50 | |
| Curcumin (tumeric) | 35 | |
| Co Q 10 | 10 | |
| Pomegranate Extract (5% Ellagic Acid) | 5 | |
| Phosphatidylcholine (from lecithin) | 1500 | mcg |

Pure Skin Clarifying Supplement

| | Amount Per Serving (mg, | |
|---|---|---|
| Ingredient | unless otherwise specified) | |
| Vitamin A (as Palmitate) | 4000 | IU |
| Beta Carotene | 2500 | IU |
| Vitamin B-1 (Thiamine HCl) | 25 | |
| Vitamin B-2 (Riboflavin) | 25 | |
| Vitamin B-3 (Niacin) | 50 | |
| Vitamin B-5 (Pantothenic Acid) | 25 | |
| Vitamin B-6 (Pyridoxine HCl) | 50 | |
| Biotin | 300 | mcg |
| Vitamin C (Calcium 60% & Zinc | 300 | |
| Ascorbate 40%) | | |
| Folic Acid | 400 | mcg |
| Vitamin E Natural | 400 | IU |
| Calcium (Ascorbate) | 62 | mg |
| Magnesium (Oxide) | 200 | mg |
| Zinc (Ascorbate) | 15 | |
| Glucosamine HCl | 65 | |
| L-Lysine HCl | 250 | |
| L-Glycine | 250 | |
| L-Proline | 500 | |
| Alpha Lipoic Acid | 50 | |
| Silica (Derived from 400 mg Horsetail | 28 | |
| Leaf Extract) | | |
| Grape Seed Extract (38.4%) | 50 | |
| Selenium (chelate) | 200 | mcg |
| Lecithin | 75 | |
| Essential Fatty Acid Complex | 150 | |
| Burdock Root Powder | 83 | |
| Yellowlock Powder | 97 | |

Youth Builder Supplement

| | Amount Per Serving (mg, | |
|---|---|---|
| Ingredient | unless otherwise specified) | |
| Vitamin A (Palmitate) | 4000 | IU |
| Vitamin E (d-alpha tocopherol succinate) | 100 | IU |
| Vitamin C (from ascorbic acid | 400 | |
| & magnesium ascorbate) | | |
| Vitamin B-3 (Niacinamide) | 80 | |
| Vitamin B-6 (Pyridoxine HCl) | 20 | |
| Zinc (from opti-zinc ™ 24 mg) | 6 | |
| Magnesium (ascorbate) | 12 | |
| Copper (sebacate) | 1.6 | |
| Selenium (chelate) | 80 | mcg |
| Glucosamine Sulfate | 1200 | |
| L-Proline | 360 | |
| L-Lysine (HCl) | 320 | |
| N-Acetyl D-Glucosamine | 160 | |
| Essential Fatty Acids Complex | 150 | |
| Beet Root Powder | 135 | |
| N-Acetyl Cysteine | 120 | |
| Quercetin | 80 | |
| Phosphatidyl choline | 45 | |
| L-Glycine | 45 | |
| Inositol | 45 | |
| Curcumin | 45 | |
| Grape Seed Extract | 30 | |

Pomphenol Sunguard Supplement

| | Amount Per Serving (mg, unless |
|---|---|
| Ingredient | otherwise specified) |
| Pomegranate (Punica Granatum) | 15 |

A.M. and P.M. Wrinkle Recovery Dietary Supplement Pack

Amount Per Serving (mg,
unless otherwise specified)

| Ingredient | A.M. Pack | | P.M. Pack | |
|---|---|---|---|---|
| Vitamin A | 2000 | IU | 2000 | IU |
| Vitamin C | 260 | | 260 | |
| Vitamin E | 103 | IU | 100 | IU |
| Niacin | 40 | | 40 | |
| Vitamin B6 | 10 | | 10 | |
| Magnesium | 6 | | 6 | |
| Zinc | 8 | | 8 | |
| Selenium | 115 | mcg | 115 | mcg |
| Copper | 1.8 | | 1.8 | |
| Manganese | 2 | | 2 | |
| Potassium | 63 | | 63 | |
| Flaxseed Oil | 816 | | 150 | |
| Alpha Linolenic Acid | 333 | | | |
| Linoleic Acid | 86 | | | |
| Glucosamine Sulfate | 685 | | 685 | |
| Fish Oil | 666 | | | |
| EPA (Eicoasapentanoic Acid) | 200 | | | |
| DHA (Docosahexaenoic Acid) | 133 | | | |
| Borage Oil | 400 | | | |
| Gamma Linolenic Acid | 80 | | | |
| L-Lysine Hydrochloride | 256 | | 256 | |
| L-Lysine | 205 | | 205 | |
| L-Proline | 180 | | 180 | |
| Chicken Collagen | 125 | | 125 | |
| Choline Bitartrate | 106 | | 106 | |
| Choline | 50 | | 50 | |
| N-Acetyl D-Glucosamine | 80 | | 80 | |
| Beet (root) | 67.5 | | 67.5 | |
| L-Glycine | 67.5 | | 67.5 | |
| N-Acetyl Cysteine | 60 | | 60 | |
| Tumeric Extract (root) | 42 | | 42 | |
| Curcumin | 40 | | 40 | |
| Quercetin | 40 | | 40 | |
| Aloe Vera Extract (leaf) | 30 | | 30 | |
| Soy Lecithin | 25 | | 25 | |
| Inositol | 22.5 | | 22.5 | |
| Pomegranate Extract (fruit) | 17.5 | | 17.5 | |
| Grape Extract (seed) | 15 | | 15 | |
| Rosemary Extract (leaf) | 6 | | | |
| Coenzyme Q 10 | 5 | | 5 | |

| | | Increase in | |
|---|---|---|---|
| | | Intracellular | |
| | | Water Content | |
| Patient No. | Age | (% increase) | Clinical Observations |
| 1. | 27 | 1.0 | Increased energy; improved health; clearer |
| | | | thinking |
| 2. | 25 | 1.4 | Increased energy; decreased Premenstrual |
| | | | Syndrome (PMS) and cellulite; reduction in |
| | | | bodyfat by 1.6% |
| 3. | 45 | 1.2 | Mentally clearer; lower blood pressure; less |
| | | | stress |
| 4. | 29 | 1.3 | Increased skin hydration; less PMS; improved |
| | | | endurance |
| 5. | 58 | 1.0 | Reduction in bodyfat by 2.0%; improved |
| | | | energy, skin hydration and oral health (teeth and |
| | | | gums) |
| 6. | 45 | 1.8 | Increase in skin hydration, firmness, and tone; |
| | | | increase in energy; less cellulite, stretch marks, |
| | | | and spider veins; less PMS symptoms |
| 7. | 61 | 0.6 | Increased hydration, energy, hair and nail |
| | | | growth; bodyfat decreased by 1.7% |
| 8. | 42 | 1.0 | Increased energy; reduced cellulite; reduced |
| | | | bodyfat by 1.1%; firmer and more hydrated |
| | | | skin; increase in hair growth; less PMS |
| 9. | 32 | 1.0 | Increased firmness, hydration and smoothness of |
| | | | skin; increased energy; improved sleep; |
| | | | reduction in cramps during menstruation; |
| | | | increased hair growth; increased endurance; |
| | | | reduction in bodyfat by 1.4% |
| 10. | 60 | 2.5 | Increase in firmness, hydration and smoothness |
| | | | of skin; improved energy, health, hair growth, |
| | | | nail growth; clearer thinking; decreased |
| | | | menopause symptoms; improved cholesterol |
| | | | levels; decrease in bodyfat by 3% |
| 11. | 46 | 0.3 | Improved sleep; more calm; skin hydration |
| | | | increased |
| 12. | 82 | 1.4 | Client was ill with a bronchial infection and |
| | | | reported that he recovered quickly and his energy |
| | | | level was greatly improved; client made less trips |
| | | | to the restroom; improved blood pressure and |
| | | | mental clarity |
| 13. | 63 | 0.9 | Increased energy; improved hair growth and skin |
| | | | hydration; more relaxed; improved digestion; |
| | | | reduced blood pressure; reduced bodyfat by 1.5% |
| 14. | 25 | 2.8 | Improved hair growth; healthier hair; less PMS |
| 15. | 28 | 0.4 | Improved energy; increase in skin hydration; |
| | | | decrease in bodyfat by 2.7% |
| 16. | 29 | 5.4 | Decrease in inches in the hips and thighs; weight |
| | | | decrease of 6.5%; bodyfat reduced by 15%; |
| | | | increased energy; joints are less painful; blood |
| | | | pressure decreased |
| 17. | 25 | 2.4 | Increased energy; increase hair growth and |
| | | | hydration in skin; decreased bodyfat by more |
| | | | than 8% |
| 18. | 67 | 2.0 | Increased hair growth; increased hydration and |
| | | | firmness of skin; decrease in bodyfat by 1.4%; |
| | | | sleeping better |
| 19. | 73 | 2.4 | Improved energy and digestion; less dryness in |
| | | | skin; lower blood pressure |
| 20. | 39 | 1.8 | Redness of skin reduced; skin hydration |
| | | | increased; energy level improved; less skin |
| | | | irritation; decreased bodyfat percentage by 1.2% |
| 21. | 65 | 2.5 | Increased energy; clearer thinking; decrease in |
| | | | bodyfat by 2.7% |
| 22. | 48 | 0.7 | Decreased insulin intake; body fat decreased by |
| | | | 3.4%; increased energy and skin hydration; less |
| | | | swelling; healthier hair |
| 23. | 77 | 1.3 | Improved digestion; more energy |
| 24. | 51 | 3.1% | Increased energy, less stressed; clearer thinking; |
| | | | increased hair and nail growth; better sleep; |
| | | | Decrease in body fat by 4.3%; loss of 3.5 |
| | | | pounds |
| 25. | 65 | 1.3% | Increased energy, endurance, and hair growth; |
| | | | improved digestion and thinking; improved skin |
| | | | hydration, decreased bodyfat by 1.3% |
| 26. | 37 | 0.5 | Decreased eczema by 60%; increased energy and |
| | | | skin hydration; improved sleep |
| 27. | 84 | 1.2 | Increased energy and hydration of skin |
| 28. | 68 | 0.7 | Decrease in edema; improved sleep |
| 29. | 54 | 1.2 | Increased energy; increased skin hydration; |
| | | | decreased bodyfat by 2% |
| 30. | 27 | 0.3 | Decrease in extracellular water content by 1.3%; |
| | | | loss of 0.5 inches on each thigh, decreased |
| | | | weight by 3 pounds; increase in energy; firmer, |
| | | | smoother, and more hydrated skin |
| 31. | 16 | 1.2% | Decrease in acne by 30%; decrease in redness; |
| | | | increases in hydration, firmness, energy, and self |
| | | | esteem |
| 32. | 32 | 1.3 | Decrease in bladder irritation; increase in energy, |
| | | | firmness of skin, hydration, and hair growth |
| 33. | 72 | 2.4 | Increase in energy; firmer, more hydrated skin; |
| | | | less joint pain |
| 34. | 52 | 1.0 | Increase in energy and mental acuity; less joint |
| | | | pain; decrease in hair falling out; more hydration |
| | | | to skin |
| 35. | 32 | 1.2 | Increase in energy; better immunity; less PMS |
| | | | symptoms; decrease in stretch marks |
| 36. | 49 | 1.0 | Increased energy and skin hydration |
| 37. | 30 | 1.0 | Increase in hair growth and nail growth; healthier |
| | | | hair; increase in energy; better thinking |
| 38. | 41 | 2.0 | Increase in energy; improved blood count; |
| | | | increased hair growth, skin hydration, and |
| | | | mental acuity |
| 39. | 55 | 1.3 | Increased energy, mental acuity; and motivation; |
| | | | firmer, more hydrated skin; improved gum |
| | | | health |
| 40. | 56 | 2.4 | Increase in energy; more color to skin; increase |
| | | | in hydration and firmness to skin; improved |
| | | | digestion; decrease in fat by 1% |
| 41. | 49 | 2.1 | Increase in new hair growth; less hair falling out; |
| | | | more energy; better mental acuity (in studying |
| | | | for the bar) |
| 42. | 53 | 2.5 | Energy increase; improved blood pressure; |
| | | | firmer, more hydrated skin; improved digestion |
| 43. | 67 | 1.4 | Increased hydration in skin and hair; increased |
| | | | energy |
| 44. | 50 | 3.3 | Increased energy; healthier skin; better sleep; |
| | | | less irritable |
| 45. | 55 | 2.5 | Increased energy; skin more hydrated and less |
| | | | irritated; increased hair growth; blood pressure |
| | | | improved |
| 46. | 43 | 3.2 | Less pigmentation; improved skin hydration and |
| | | | firmness of skin; improved digestion |
| 47. | 56 | 0.5 | Increased energy, hair growth; stronger nails; |
| | | | improved hydration and mental acuity |
| 48. | 32 | 0.9 | Improved hair growth; less damage to skin when |
| | | | in the sun; increased hydration; firmer skin; |
| | | | decreased pigmentation |
| 49. | 40 | 1.1 | Increased energy; less PMS symptoms; less joint |
| | | | pain; increased motivation and mental acuity |
| 50. | 44 | 2.3 | Increased energy, hair and nail growth; less dry |
| | | | irritated skin; improved digestion and sleep |
| 51. | 37 | 1.0 | Improved energy, hydration, and hair growth; |
| | | | less pigmentation; less headaches |
| 52. | 18 | 0.5 | Reduced acne and inflammation; decreased pore |
| | | | size; less PMS symptoms; better digestion |
| 53. | 42 | 0.9 | Increased energy, hydration, skin firmness; |
| | | | improved digestion |
| 54. | 41 | 0.6 | Decreased redness and inflammation; improved |
| | | | hair and nail growth |
| 55. | 28 | 3.0 | Increased energy, hair growth; less hair falling |
| | | | out; improved digestion |
| 56. | 60 | 1.0 | Increase in skin hydration; increased glow to |
| | | | skin; increased hair growth |
| 57. | 62 | 2.1 | Increase in hydration; less irritated; more energy; |
| | | | better sleep |
| 58. | 32 | 2.2 | Decrease in eczema on the face and around the |
| | | | eyes; improved energy; less skin irritation when |
| | | | shaving; lower blood pressure |
| 59. | 45 | 1.7 | Increase hydration and firmness of skin; |
| | | | improved sleep and digestion |
| 60. | 66 | 0.8 | Less irritation accompanying sun exposure; |
| | | | increased hydration and hair growth; less |
| | | | stressed. |
| 61. | 55 | 1.9 | Increased hydration and firmness; skin less |
| | | | irritated; improved mental acuity |
| 62. | 67 | 1.6 | Improved hydration and firmness; less |
| | | | pigmentation; less irritable |
| 63. | 40 | 1.1 | Increased hydration and hair growth, less skin |
| | | | lesions; improved mental acuity |
| 64. | 42 | 3.2 | Improved energy, hydration of skin, and |
| | | | motivation; less stressed |
| 65. | 43 | 1.6 | Increased hydration, firmness, energy, and hair |
| | | | growth; improved sleep. |
| 66. | 37 | 6.9 | Decreased eczema, inflammation; increased |
| | | | energy, hydration; improved mood and digestion |
| 67. | 34 | 1.2 | Increased energy, glow to skin, hydration; |
| | | | improved sleep; less hot flashes |
| 68. | 34 | 1.8 | Increased energy, glow to skin, hydration, skin |
| | | | firmness; improved mood |
| 69. | 38 | 1.2 | Increased hair and nail growth; increased energy; |
| | | | better sleep and less PMS symptoms |
| 70. | 32 | 2.6 | Decreased eczema, improved skin hydration and |
| | | | glow; no itching or cracking; improved sleep and |
| | | | digestion |
| 71. | 56 | 1.3 | Increased hydration, energy, and mental acuity |
| 72. | 45 | 1.6 | Increased skin firmness, hydration, and skin |
| | | | tone; decrease in menstrual cramps and PMS |
| | | | symptoms; reduction in bodyfat by 1.6% |
| 73. | 60 | 1.4 | Decreased swelling in right arm; increased |
| | | | energy and sleep |
| 74. | 43 | 1.3 | Increased hydration and firmness; decreased |
| | | | cellulite; bodyfat reduction of 2.0%; improved |
| | | | digestion and energy; motivation to improve diet |
| 75. | 34 | 0.7 | Increase in energy; improved digestion; liver |
| | | | enzymes normal for the first time in 2 years; |
| | | | improved hydration and less skin allergies |
| 76. | 40 | 4.7 | Increased hydration, firmness; less wrinkles; |
| | | | improved energy and well being; better sleep |
| 77. | 48 | 1.7 | Increased firmness, hydration, smoothness and |
| | | | tone of skin; improved energy |
| 78. | 67 | 0.3 | Increased energy; hydration, and firmness of skin |
| 79. | 62 | 0.3 | Increased energy, hydration; and mental acuity |
| 80. | 32 | 0.3 | Decrease in hair loss; new hair growth; less |
| | | | stressed |

These case studies show that an increase in intracellular water content is correlated with an improvement in a variety of dermatological and health related conditions including acne, eczema, rosacea, spider veins, high blood pressure, and diabetes.

Other representative pharmaceutical compositions useful in the methods of the invention include:
(i) Murad Environmental Shield Vitamin C Infusion Treatment Gel (which contains Glycerin, Glyceryl Polymethacrylate, Cyanocobalamin, Retinyl Palmitate, Beta-Carotene, Ranunculus Ficaria Extract, Rice Amino Acids, Lysine Lauroyl Methionate, Zinc Aspartate, and Chitosan Ascorbate);
(ii) Murad Environmental Shield Essential C Daily Renewal Complex (which contains Cyclomethicone, Ascorbic Acid, Dimethicone Crosspolymer, C₁₂₋₁₅ Alkyl Ethylhexanoate, Di-C₁₂₋₁₁₅ Alkyl Fumarate, Tocopheryl Acetate, Retinol, Bisabolol, Allantoin, Lysine Lauroyl Methionate, Rice Amino Acids, Zinc Aspartate, Chitosan Ascorbate, Retinyl Palmitate, Cyanocobalamin, Beta-Carotene, Ranunculus Ficaria Extract, Glycine, Glutamic Acid, Caprylic/Capric Triglyceride, Vegetable Oil (Olus), Limonene, Carthamus Tinctorius (Safflower) Seed Oil, Citrus Aurantium Dulcis (Orange) Oil, Citrus Nobilis (Mandarin Orange) Peel Oil, Ocimum Basilicum (Basil) Oil, Citrus Grandis (Grapefruit) Peel Oil, and Ferula Galbaniflua (Galbanum) Resin O);
(iii) Murad Essential C Age Proof Eye Cream (which contains Octinoxate (7.5%), Octisalate (3.0%), Oxybenzone (2.0%) (as active ingredients), Water (Aqua), Butylene Glycol, Bis-Diglyceryl Polyacyladipate-2, Caprylic/Capric/Stearic Triglyceride, Shorea Stenoptera Butter, PEG-100 Stearate, Glyceryl Stearate, Cetyl Alcohol, Silica, Stearic Acid, Lysine Lauroyl Methionate, Rice Amino Acids, Zinc Aspartate, Chitosan Ascorbate, Retinol, Glycine Soja (Soybean) Oil, Persea Gratissima (Avocado) Oil Unsaponifiables, Cimicifuga Racemosa Root Extract, Caffeine, Siloxanetriol Alginate, Centella Asiatica Extract, Methylsilanol Mannuronate, Dimethicone, Phospholipids, Tocopheryl Acetate, Retinyl Palmitate, Ascorbyl Palmitate, Panthenol, Sodium PCA, Cetyl Phosphate, Carbomer, Aminomethyl Propanol, Disodium EDTA, Phenoxyethanol, Methylparaben, Propylparaben, Titanium Dioxide (CI 77891), and Iron Oxides (CI 77491));
(iv) Murad Clarifying Skin Cleanser (which contains Salicylic Acid (1.5%) as the active ingredient, Water (Aqua), Cocamidopropyl Betaine, Sodium C₁₄₁₆ Olefin Sulfonate, Methyl Gluceth-20, PPG-26-Buteth-26, PEG-40 Hydrogenated Castor Oil, Butylene Glycol, Cimicifuga Racemosa Root Extract, Camellia Oleifera Leaf Extract, Triclosan Menthol, Limonene, Citrus Aurantium Amara (Bitter Orange) Oil, Cymbopogon Nardus (Citronella) Oil, Citrus Medica Limonum (Lemon) Peel Oil, Citrus Aurantifolia (Lime) Oil, Lavandula Angustifolia (Lavender) Oil, Citrus Tangerina (Tangerine) Oil, Prunus Armeniaca (Apricot) Kernel Oil, Tetrasodium EDTA, and Methylparaben);
(v) Murad Skin Perfecting Lotion (which contains Water (Aqua), Dicaprylyl Maleate, C₁₂₋₁₅ Alkyl Benzoate, Butylene Glycol, Glycerin, Sorbitan Stearate, Retinol, Caprylic/Capric Triglyceride, Arnica Montana Flower Extract, Epilobium Angustifolium Extract, Palmitoyl Hydroxypropyltrimonium Amylopectin/Glycerin Crosspolymer, Vitis Vinifera (Grape) Seed Extract, Camellia Sinensis Leaf Extract, Avena Sativa (Oat) Kernel Protein, Hydrolyzed Soy Flour, Spiraea Ulmaria Flower Extract, Honey Extract (MeI), Citrus Aurantium Dulcis (Orange) Fruit Extract, Squalane, Stearic Acid, Cetearyl Alcohol, Ceteareth-20, Talc, C₉₋₁₅ Fluoroalcohol Phosphate, Glycolipids, Sodium, Hyaluronate, Panthenol, Lecithin, Tocopherol, Magnesium Ascorbyl Phosphate, Algae Extract, Dimethicone, Sodium PCA, Allantoin, Sclerotium Gum, Carbomer, Aminomethyl Propanol, Phenoxyethanol, Methylparaben, Propylparaben, and Tetrasodium EDTA).

Various modifications of the invention in addition to those shown and described herein will be apparent to those skilled in the art from the foregoing description. Such modifications are also intended to fall within the scope of the appended claims. The foregoing disclosure includes all the information deemed essential to enable those skilled in the art to practice the claimed invention.

A number of references have been cited, the entire disclosure of which are incorporated by reference.

1. A method for treating a dermatological condition in a patient comprising:
(a) measuring the intracellular water content of the patient to provide an initial intracellular water content measurement;
(b) administering to the patient at least one active agent;
(c) re-measuring the intracellular water content of the patient to provide a post-treatment intracellular water content measurement; and
(d) repeating steps (b) and (c) until the post-treatment intracellular water content measurement is greater than the initial intracellular water content measurement.

2. The method of claim 1, wherein the at least one active agent is administered orally.

3. The method of claim 1, wherein the at least one active agent is administered topically.

4. The method of claim 1, wherein the post-treatment intracellular water content measurement is at least about 0.25 percentage units greater than the initial intracellular water content measurement.

5. The method of claim 1, wherein the post-treatment intracellular water content measurement is at least about 1 percentage unit greater than the initial intracellular water content measurement.

6. The method of claim 1, wherein the post-treatment intracellular water content measurement is at least about 5 percentage units greater than the initial intracellular water content measurement.

7. The method of claim 1, wherein the post-treatment intracellular water content measurement is at least about 10 percentage units greater than the initial intracellular water content measurement.

8. The method of claim 1, wherein the active agent is administered to the patient at least daily for at least about 1 day before re-measuring the patient's intracellular water content.

9. The method of claim 1, wherein the active agent is administered to the patient at least daily for at least about 7 days before re-measuring the patient's intracellular water content.

10. The method of claim 1, wherein the active agent is administered to the patient at least daily for at least 14 days before re-measuring the patient's intracellular water content.

11. The method of claim 1, wherein the active agent is administered to the patient at least daily for at least about 28 days before re-measuring the patient's intracellular water content.

12. The method of claim 1, further comprising taking a photographic image of the patient's skin before obtaining the initial intracellular water content measurement to provide a first photograph of the dermatological condition and taking a photograph after obtaining the post-treatment intracellular water content measurement to record a second photograph of the dermatological condition and comparing the first photograph and the second photograph to evaluate whether a further increase in the patient's intracellular water content is needed to improve the dermatological condition.

13. The method of claim 1, further comprising: (a1) measuring the extracellular water content of the patient before administering the at least one active agent to the patient to provide an initial extracellular water content measurement and (c1) then re-measuring the extracellular water content after administering the at least one active agent to the patient to provide a post-treatment extracellular water content measurement; and (d1) repeating steps (a1) and (c1) until the post-treatment extracellular water content measurement is less than the initial extracellular water content measurement.

14. The method of claim 1, further comprising: (a2) measuring the basal metabolic rate of the patient before administering the at least one active agent to the patient to provide an initial basal metabolic rate measurement and (c2) then re-measuring the basal metabolic rate after administering the at least one active agent to the patient to provide a post-treatment basal metabolic rate measurement; and (d2) repeating steps (a2) and (c2) until the post-treatment basal metabolic rate measurement is greater than the initial basal metabolic rate measurement.

15. The method of claim 1, further comprising: (a1) measuring the extracellular water content of the patient before administering the at least one active agent to the patient to provide an initial extracellular water content measurement and (a2) measuring the basal metabolic rate of the patient before administering the at least one active agent to the patient to provide an initial basal metabolic rate measurement; then (c1) re-measuring the extracellular water content after administering the at least one active agent to the patient to provide a post-treatment extracellular water content measurement and (c2) re-measuring the basal metabolic rate after administering the at least one active agent to the patient to provide a post-treatment basal metabolic rate measurement; and (d1) repeating steps (a1) and (c1) until the post-treatment extracellular water content measurement is less than the extracellular water content measurement and (d2) repeating steps (a2) and (c2) until the post-treatment basal metabolic rate measurement is greater than the initial basal metabolic rate measurement.

16. The method of claim 1, wherein the at least one active agent comprises an active agent selected from the group consisting of at least one moisturizing agent, a primary antioxidant, an amino acid, at least one amino acid, and at least one transition metal.
